SEQUENCE LISTING (77 sequences)

SEQ ID NO: 1 - protein, 107 aa, Murine
  misc_feature: TES-C21 LIGHT CHAIN
DILLTQSPAILSVSPGERVSFSCRASQSIGTNIHWYQQRTDGSPRLLIKYASESISGIPSRFSGSGSGTEFTLNINSVESEDIADYYCQQSDSWPTTFGGGTKLEIK

SEQ ID NO: 2 - protein, 107 aa, Human
  misc_feature: L16/-JK4 human light chain consensus sequence template
EIVMTQSPATLSVSPGERATLSCRASQSVSSNLAWYQQKPGQAPRLLIYGASTRATGIPARFSGSGSGTEFTLTISSLQSEDFAVYYCQQYNNWPLTFGGGTKVEIK

SEQ ID NO: 3 - protein, 123 aa, MURINE
  misc_feature: TES-C21 Heavy Chain
QVQLQQSGAELMKPGASVKISCKTTGYTFSMYWLEWVKQRPGHGLEWVGEISPGTFTTNYNEKFKAKATFTADTSSNTAYLQLSGLTSEDSAVYFCARFSHFSGSNYDYFDYWGQGTSLTVSS

SEQ ID NO: 4 - protein, 113 aa, Human
  misc_feature: DP88/JH4b human heavy chain consensus sequence template
QVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARYFDYLVQGTSLTVSS

SEQ ID NO: 5 - protein, 11 aa, ARTIFICIAL
  TES-C21 CDRL1 SEQUENCE (TABLE 1)
RASQSIGTNIH

SEQ ID NO: 6 - protein, 11 aa, ARTIFICIAL
  CDRL1 VARIANT SEQUENCE #1 (TABLE 1)
RASRSIGTNIH

SEQ ID NO: 7 - protein, 11 aa, ARTIFICIAL
  CDRL1 VARIANT SEQUENCE #2 (TABLE 1)
RASQRIGTNIH

SEQ ID NO: 8 - protein, 7 aa, ARTIFICIAL
  TES-C21 CDRL2 SEQUENCE (TABLE 1)
YASESIS

SEQ ID NO: 9 - protein, 7 aa, ARTIFICIAL
  CDRL2 VARIANT #1 (TABLE 1)
YAYESIS

SEQ ID NO: 10 - protein, 7 aa, ARTIFICIAL
  CDRL2 VARIANT #2 (TABLE 1)
YASESIY

SEQ ID NO: 11 - protein, 7 aa, ARTIFICIAL
  CDRL2 VARIANT #3 (TABLE 1)
YASESDS

SEQ ID NO: 12 - protein, 7 aa, ARTIFICIAL
  CDRL2 VARIANT #4 (TABLE 1)
YASESES

SEQ ID NO: 13 - protein, 9 aa, ARTIFICIAL
  TES-C21 CDRL3 (TABLE 1)
QQSDSWPTT

SEQ ID NO: 14 - protein, 9 aa, ARTIFICIAL
  CDRL3 VARIANT (TABLE)
AASWSWPTT

SEQ ID NO: 15 - protein, 5 aa, ARTIFICIAL
  TES-C21 CDRH1
MYWLE

SEQ ID NO: 16 - protein, 5 aa, ARTIFICIAL
  CDRH1 VARIANT #1 (TABLE 1)
WYWLE

SEQ ID NO: 17 - protein, 5 aa, ARTIFICIAL
  CDRH1 #2 (TABLE 1)
YYWLE

SEQ ID NO: 18 - protein, 17 aa, ARTIFICIAL
  TES-C21 CDRH2 (TABLE 1)
EISPGTFTTNYNEKFKA

SEQ ID NO: 19 - protein, 17 aa, ARTIFICIAL
  CDRH2 VARIANT #1 (TABLE 1)
EIEPGTFTTNYNEKFKA

SEQ ID NO: 20 - protein, 17 aa, ARTIFICIAL
  CDRH2 VARIANT #2 (TABLE 1)
EIDPGTFTTNYNEKFKA

SEQ ID NO: 21 - protein, 17 aa, ARTIFICIAL
  CDRH2 VARIANT #3 (TABLE 1)
EISPDTFTTNYNEKFKA

SEQ ID NO: 22 - protein, 17 aa, ARTIFICIAL
  CDRH2 VARIANT #4 (TABLE 1)
EISPETFTTNYNEKFKA

SEQ ID NO: 23 - protein, 17 aa, ARTIFICIAL
  CDRH2 VARIANT #5 (TABLE 1)
EISPGTFETNYNEKFKA

SEQ ID NO: 24 - protein, 17 aa, ARTIFICIAL
  CDRH2 VARIANT #6 (TABLE 1)
EIEPGTFETNYNEKFKA

SEQ ID NO: 25 - protein, 17 aa, ARTIFICIAL
  CDRH2 VARIANT #7 (TABLE 1)
EIDPGTFETNYNEKFKA

SEQ ID NO: 26 - protein, 14 aa, ARTIFICIAL
  TES-C21 CDRH3 (TABLE 1)
FSHFSGSNYDYFDY

SEQ ID NO: 27 - protein, 14 aa, ARTIFICIAL
  CDRH3 VARIANT #1 (TABLE 1)
FSHFSGMNYDYFDY

SEQ ID NO: 28 - protein, 14 aa, ARTIFICIAL
  CDRH3 VARIANT #2 (TABLE 1)
FSHFSGQNYDYFDY

SEQ ID NO: 29 - protein, 14 aa, ARTIFICIAL
  CDRH3 VARIANT #3 (TABLE 1)
FSHFTGSNYDYFDY

SEQ ID NO: 30 - protein, 23 aa, ARTIFICIAL
  FRL1 VARIANT 136
EIVMTQSPATLSVSPGERATLSC

SEQ ID NO: 31 - protein, 23 aa, ARTIFICIAL
  FRL1 VARIANT 1
DILMTQSPATLSVSPGERATLSC

SEQ ID NO: 32 - protein, 23 aa, ARTIFICIAL
  FRL1 VARIANT 2
DIVLTQSPATLSVSPGERATLSC

SEQ ID NO: 33 - protein, 23 aa, ARTIFICIAL
  FRL1 VARIANT 4
DILLTQSPATLSVSPGERATLSC

SEQ ID NO: 34 - protein, 23 aa, ARTIFICIAL
  FRL1 VARIANT 13
EIVLTQSPATLSVSPGERATLSC

SEQ ID NO: 35 - protein, 23 aa, ARTIFICIAL
  FRL1 VARIANT 18
EILLTQSPATLSVSPGERATLSC

SEQ ID NO: 36 - protein, 23 aa, ARTIFICIAL
  FRL1 VARIANT 25
DIVMTQSPATLSVSPGERATLSC

SEQ ID NO: 37 - protein, 23 aa, ARTIFICIAL
  FRL1 VARIANT 27
EILMTQSPATLSVSPGERATLSC

SEQ ID NO: 38 - protein, 15 aa, ARTIFICIAL
  FRL2 VARIANT 136
WYQQKPGQAPRLLIY

SEQ ID NO: 39 - protein, 15 aa, ARTIFICIAL
  FRL2 VARIANT 1
WYQQKPGQAPRLLIK

SEQ ID NO: 40 - protein, 32 aa, ARTIFICIAL
  FRL3 VARIANT 136
GIPARFSGSGSGTEFTLTISSLQSEDFAVYYC

SEQ ID NO: 41 - protein, 32 aa, ARTIFICIAL
  FRL3 VARIANT 1
GIPSRFSGSGSGTEFTLTISSLQSEDFAVYYC

SEQ ID NO: 42 - protein, 32 aa, ARTIFICIAL
  FRL3 VARIANT 13
GIPARFSGSGSGTEFTLTISSLQSEDFADYYC

SEQ ID NO: 43 - protein, 32 aa, ARTIFICIAL
  FRL3 VARIANT 18
GIPSRFSGSGSGTEFTLTISSLQSEDFADYYC

SEQ ID NO: 44 - protein, 10 aa, ARTIFICIAL
  FRL4 VARIANT
FGGGTKVEIK

SEQ ID NO: 45 - protein, 30 aa, ARTIFICIAL
  FRH1 VARIANT 136
QVQLVQSGAEVMKPGSSVKVSCKASGYTFS

SEQ ID NO: 46 - protein, 30 aa, ARTIFICIAL
  FRH1 VARIANT 2
QVQLVQSGAEVKKPGSSVKVSCKASGYTFS

SEQ ID NO: 47 - protein, 14 aa, ARTIFICIAL
  FRH2 VARIANT 136
WVRQAPGHGLEWMG

SEQ ID NO: 48 - protein, 14 aa, ARTIFICIAL
  FRH2 VARIANT 2
WVRQAPGQGLEWMG

SEQ ID NO: 49 - protein, 14 aa, ARTIFICIAL
  FRH2 VARIANT 8
WVRQAPGQGLEWVG

SEQ ID NO: 50 - protein, 14 aa, ARTIFICIAL
  FRH2 VARIANT 21
WVRQAPGHGLEWVG

SEQ ID NO: 51 - protein, 32 aa, ARTIFICIAL
  FRH3 VARIANT 136
RVTFTADTSTSTAYMELSSLRSEDTAVYYCAR

SEQ ID NO: 52 - protein, 32 aa, ARTIFICIAL
  FRH3 VARIANT 1
RATFTADTSTSTAYMELSSLRSEDTAVYYCAR

SEQ ID NO: 53 - protein, 32 aa, ARTIFICIAL
  FRH3 VARIANT 43
RVTITADTSTSTAYMELSSLRSEDTAVYYCAR

SEQ ID NO: 54 - protein, 32 aa, ARTIFICIAL
  FRH3 VARIANT 103
RATITADTSTSTAYMELSSLRSEDTAVYYCAR

SEQ ID NO: 55 - protein, 11 aa, ARTIFICIAL
  FRH4 VARIANT 136
WGQGTLVTVSS

SEQ ID NO: 56 - protein, 19 aa, Bacteriophage M13mp18
  misc_feature: Gene III signal Sequence
MEWSGVFMFLLSVTAGVHS

SEQ ID NO: 57 - protein, 107 aa, ARTIFICIAL
  LIGHT CHAIN VARIABLE REGION OF CLONE 136
EIVMTQSPATLSVSPGERATLSCRASQSIGTNIHWYQQKPGQAPRLLIYYASESISGIPARFSGSGSGTEFTLTISSLQSEDFAVYYCQQSDSWPTTFGGGTKVEIK

SEQ ID NO: 58 - protein, 106 aa, ARTIFICIAL
  LIGHT CHAIN CONSTANT REGION OF CLONE 136
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 59 - protein, 123 aa, ARTIFICIAL
  HEAVY CHAIN VARIABLE REGION OF CLONE 136
QVQLVQSGAEVMKPGSSVKVSCKASGYTFSMYWLEWVRQAPGHGLEWMGEISPGTFTTNYNEKFKARVTFTADTSTSTAYMELSSLRSEDTAVYYCARFSHFSGSNYDYFDYWGQGTLVTVSS

SEQ ID NO: 60 - protein, 330 aa, ARTIFICIAL [sequence omitted]

SEQ ID NO: 61 - protein, 107 aa, ARTIFICIAL
  LIGHT CHAIN VARIABLE REGION OF CLONE CL-2C
EIVMTQSPATLSVSPGERATLSCRASQSIGTNIHWYQQKPGQAPRLLIYYASESISGIPARFSGSGSGTEFTLTISSLQSEDFAVYYCQQSWSWPTTFGGGTKVEIK

SEQ ID NO: 62 - protein, 123 aa, ARTIFICIAL
  HEAVY CHAIN VARIABLE REGION OF CLONE CL-2C
QVQLVQSGAEVMKPGSSVKVSCKASGYTFSWYWLEWVRQAPGHGLEWMGEIDPGTFTTNYNEKFKARVTFTADTSTSTAYMELSSLRSEDTAVYYCARFSHFSGSNYDYFDYWGQGTLVTVSS

SEQ ID NO: 63 - protein, 107 aa, ARTIFICIAL; identical to SEQ ID NO: 61
  LIGHT CHAIN VARIABLE REGION OF CLONE CL-5I

SEQ ID NO: 64 - protein, 123 aa, ARTIFICIAL
  HEAVY CHAIN VARIABLE REGION OF CLONE CL-5I
QVQLVQSGAEVMKPGSSVKVSCKASGYTFSMYWLEWVRQAPGHGLEWMGEIDPGTFETNYNEKFKARVTFTADTSTSTAYMELSSLRSEDTAVYYCARFSHFSGSNYDYFDYWGQGTLVTVSS

SEQ ID NO: 65 - protein, 107 aa, ARTIFICIAL; identical to SEQ ID NO: 61
  LIGHT CHAIN VARIABLE REGION OF CLONE CL-5A

SEQ ID NO: 66 - protein, 123 aa, ARTIFICIAL
  HEAVY CHAIN VARIABLE REGION OF CLONE CL-5A
QVQLVQSGAEVMKPGSSVKVSCKASGYTFSWYWLEWVRQAPGHGLEWMGEIEPGTETTNYNEKFKARVTFTADTSTSTAYMELSSLRSEDTAVYYCARFSHFSGSNYDYFDYWGQGTLVTVSS

SEQ ID NO: 67 - protein, 107 aa, ARTIFICIAL; identical to SEQ ID NO: 61
  LIGHT CHAIN VARIABLE REGION OF CLONE CL-2B

SEQ ID NO: 68 - protein, 123 aa, ARTIFICIAL
  HEAVY CHAIN VARIABLE REGION OF CLONE CL-2B
QVQLVQSGAEVMKPGSSVKVSCKASGYTFSYYWLEWVRQAPGHGLEWMGEIDPGTFTTNYNEKFKARVTFTADTSTSTAYMELSSLRSEDTAVYYCARFSHFSGSNYDYFDYWGQGTLVTVSS

SEQ ID NO: 69 - protein, 107 aa, ARTIFICIAL; identical to SEQ ID NO: 61
  LIGHT CHAIN VARIABLE REGION OF CLONE CL-1136-2C

SEQ ID NO: 70 - protein, 123 aa, ARTIFICIAL
  HEAVY CHAIN VARIABLE REGION OF CLONE CL-1136-2C
QVQLVQSGAEVKKPGSSVKVSCKASGYTFSMYWLEWVRQAPGQGLEWMGEISPGTFTTNYNEKFKARVTFTADTSTSTAYMELSSLRSEDTAVYYCARFSHFSGSNYDYFDYWGQGTLVTVSS

SEQ ID NO: 71 - protein, 108 aa, Homo sapiens
DSNPRGVSAYLSRPSPFDLFIRKSPTITCLVVDLAPSKGTVNLTWSRASGKPVNHSTRKEEKQRNGTLTVTSTLPVGTRDWIEGETYQCRVTHPHLPRALMRSTTKTS

SEQ ID NO: 72 - protein, 12 aa, ARTIFICIAL
  PEPTIDE DERIVED FROM CH3 DOMAIN OF IGE
NPRGVSXYXXRX

SEQ ID NO: 73 - protein, 12 aa, ARTIFICIAL
  PEPTIDE DERIVED FROM CH3 DOMAIN OF IGE
NPRGVSAYLSRP

SEQ ID NO: 74 - protein, 9 aa, ARTIFICIAL
  PEPTIDE DERIVED FROM CH3 DOMAIN OF IGE
LPRALXRSX

SEQ ID NO: 75 - protein, 9 aa, ARTIFICIAL
  PEPTIDE DERIVED FROM CH3 DOMAIN OF IGE
LPRALMRST

SEQ ID NO: 76 - protein, 12 aa, ARTIFICIAL
  PEPTIDE DERIVED FROM CH3 DOMAIN OF IGE
HPHLPRALMRST

SEQ ID NO: 77 - protein, 12 aa, ARTIFICIAL
  PEPTIDE DERIVED FROM CH3 DOMAIN OF IGE
LPRALMRSTTKT

(19) United States
(12) Patent Application Publication
(10) Pub. No.: US 2011/0200604 A1
(43) Pub. Date: Aug. 18, 2011

(54) IDENTIFICATION OF NOVEL IgE EPITOPES

(75) Inventors: Sanjaya Singh, Houston, TX (US); Danyang Huang, Houston, TX (US); Sek Chung Michael Fung, Houston, TX (US)

(73) Assignee: Tanox, Inc.

(21) Appl. No.: 12/879,757

(22) Filed: Sep. 10, 2010

Related U.S. Application Data
(63) Continuation of application No. 10/901,736, filed on Jul. 29, 2004, now abandoned.
Application No. 10/901,736 is a continuation-in-part of application No. PCT/US04/02892, filed on Feb. 2, 2004.
Application No. PCT/US04/02892 is a continuation-in-part of application No. PCT/US04/02894, filed on Feb. 2, 2004.
(60) Provisional application No. 60/444,229, filed on Feb. 1, 2003.

(51) Int. Cl. A61K 39/395; C07K 16/42; C07K 16/46; A61P 11/06; A61P 17/00; A61P 11/00; C40B 30/04

(52) U.S. Cl. 424/139.1; 530/387.9; 530/387.3; 530/391.3; 506/9

(57) ABSTRACT

The present invention relates to novel peptide epitopes derived from the CH3 domain of IgE which are recognized by high affinity antibodies that specifically bind IgE. These novel peptides may be used for both active immunization of a subject by administering these peptides to generate high affinity antibodies in a subject, as well as for generating high affinity anti-IgE antibodies in non-human hosts that specifically bind to these regions of IgE for passive immunization of a subject.

### CROSS RELATED APPLICATIONS

The present application claims priority to PCT Application No. PCT/US04/02892 and PCT/US041/02894 filed Feb. 2, 2004, which claim priority to U.S. Provisional Application 60/444,229 filed Feb. 1, 2003, all of which are incorporated by reference herein.

### BACKGROUND OF THE INVENTION

Allergy is a hypersensitive state induced by an exaggerated immune response to a foreign agent, such as an allergen. Immediate (type I) hypersensitivity, characterized by allergic reactions immediately following contact with the allergen, is mediated via B cells and is based on antigen-antibody reactions. Delayed hypersensitivity is mediated via T cells and based on mechanisms of cellular immunity. In recent years, the term “allergy” has become more and more synonymous with type I hypersensitivity.

Immediate hypersensitivity is a response based on the production of antibodies of the immunoglobulin class E (IgE antibodies) by B cells which upon exposure to an allergen differentiate into antibody secreting plasma cells. The IgE induced reaction is a local event occurring at the site of the allergen's entry into the body, i.e. at mucosal surfaces and/or at local lymph nodes. Locally produced IgE will first sensitize local mast cells, i.e. IgE antibodies bind with their constant regions to FCε receptors on the surface of the mast cells, and then “spill-over” IgE enters the circulation and binds to receptors on both circulating basophils and tissue-fixed mast cells throughout the body. When the bound IgE is subsequently contacted with the allergen, the FCε receptors are crosslinked by binding of the allergen causing the cells to degranulate and release a number of anaphylactic mediators such as histamine, prostaglandins, leukotrienes, etc. It is the release of these substances which is responsible for the clinical symptoms typical of immediate hypersensitivity, namely contraction of smooth muscle in the respiratory tract or the intestine, the dilation of small blood vessels and the increase in their permeability to water and plasma proteins, the secretion of mucus resulting, e.g in allergic rhinitis, atopic excema and asthma, and the stimulation of nerve endings in the skin resulting in itching and pain. In addition, the reaction upon second contact with the allergen is intensified because some B cells form a “memory pool” of surface IgE positive B cells (sIgE⁺ B cells) after the first contact with the allergen by expressing IgE on the cell surface.

There are two major receptors for IgE, the high affinity receptor FcεRI and the low-affinity receptor FcεRII. FcεRI is predominantly expressed on the surface of mast cells and basophils, but low levels of FcεRI can also be found on human Langerhan's cells, dendritic cells, and monocytes, where it functions in IgE-mediated allergen presentation. In addition, FcεRI has been reported on human eosinophils and platelets (Hasegawa, S. et. al., Hematopoiesis, 1999, 93:2543-2551). FcεRI is not found on the surface of B cells, T cells, or neutrophils. The expression of FcεRI on Langerhan's cells and dermal dendritic cells is functionally and biologically important for IgE-bound antigen presentation in allergic individuals (Klubal R. et al., J. Invest. Dermatol. 1997, 108 (3):336-42).

The low-affinity receptor, FcεRII (CD23) is a lectin-like molecule comprising three identical subunits with head structures extending from a long α-helical coiled stalk from the cellular plasma membrane (Dierks, A. E. et al., J. Immunol. 1993, 150:2372-2382). Upon binding to IgE, FcεRII associates with CD21 on B cells involved in the regulation of synthesis of IgE (Sanon, A. et al., J. Allergy Clin. Immunol. 1990, 86:333-344, Bonnefoy, J. et al., Eur. Resp. J. 1996, 9:63s-66s). FcεRII has long been recognized for allergen presentation (Sutton and Gould, 1993, Nature, 366:421-428). IgE bound to FcεRII on epithelial cells is responsible for specific and rapid allergen presentation (Yang, P. P., J. Clin. Invest., 2000, 106:879-886). FcεRII is present on several cell types, including B-cells, eosinophils, platelets, natural killer cells, T-cells, follicular dendritic cells, and Langerhan's cells.

The structural entities on the IgE molecule that interact with FcεRI and FcεRII have also been identified. Mutagenesis studies have indicated that the CH3 domain mediates IgE interaction with both FcεRI (Presta et al., J. Biol. Chem. 1994, 269:26368-26373; Henry A. J. et al., Biochemistry, 1997, 36:15568-15578) and FcεRII (Sutton and Gould, Nature, 1993, 366: 421-428; Shi, J. et al., Biochemistry, 1997, 36:2112-2122). The binding sites for both high- and low-affinity receptors are located symmetrically along a central rotational axis through the two CH3 domains. The FcεRI-binding site is located in a CH3 domain on the outward side near the junction of the CH2 domain, whereas the FcεRII-binding site is on the carboxyl-terminus of CH3.

A promising concept for the treatment of allergy involves the application of monoclonal antibodies, which are IgE isotype-specific and are thus capable of binding IgE. This approach is based on the inhibition of allergic reactions by downregulating the IgE immune response, which is the earliest event in the induction of allergy and provides for the maintenance of the allergic state. As the response of other antibody classes is not affected, both an immediate and a long lasting effect on allergic symptoms is achieved. Early studies of human basophil density showed a correlation between the level of IgE in the plasma of a patient and the number of FcεRI receptors per basophil (Malveaux et al., J. Clin. Invest., 1978, 62:176). They noted that the FcεRI densities in allergic and non-allergic persons range from 10⁴ to 10⁶ receptors per basophil. Later it was shown that treatment of allergic diseases with anti-IgE decreased the amount of circulating IgE to 1% of pretreatment levels (MacGlashan et al., J. Immunol., 1997, 158:1438-1445). MacGlashan analyzed serum obtained from patients treated with whole anti-IgE antibody, which binds free. IgE circulating in the serum of the patient. They reported that lowering the level of circulating IgE in a patient resulted in a lower number of receptors present on the surface of basophils. Thus, they hypothesized that FcεRI density on the surface of basophils and mast cells is directly or indirectly regulated by the level of circulating IgE antibody.

More recently, WO 99/62550 disclosed the use of IgE molecules and fragments, which bind to FcεRI and FcεRII IgE binding sites to block IgE binding to receptors. However, effective therapies that lack deleterious side effects for the management of these allergic diseases are limited. One therapeutic approach to treating allergic diseases involved using humanized anti-IgE antibody to treat allergic rhinitis and asthma (Come, J. et al., J. Clin. Invest. 1997, 99:879-887; Racine Poon, A. et al., Clin. Pharmcol. Ther. 1997, 62:675-690; Fahy, J. V. et al., Am. J. Resp. Crit. Care Med. 1997, 155:1824-1834; Boulet, L. P. et al., Am. J. Resp. Crit. Care Med., 1997, 155:1835-1840; Milgrom, E. et al., N. Engl. J. Med., 1999, 341:1966-1973). These clinical data demonstrate that inhibition of IgE binding to its receptors is an effective approach to treating allergic diseases.

Antibodies suitable as anti-allergic agents should react with surface IgE positive B cells which differentiate into IgE producing plasma cells, so that they can be used to functionally eliminate those B cells. However, antibodies to IgE in principle may also induce mediator release from IgE sensitized mast cells by crosslinking the Fce receptors, thus antagonizing the beneficial effect exerted on the serum IgE and sIgE⁺ B cell level. One of the potentially dangerous problems with developing anti-IgE therapies is the possibility of IgE-crosslinking caused by the therapeutic antibody binding to IgE already bound to the high affinity receptor and triggering histamine release resulting in a potentially anaphylactic reaction.

Therefore, antibodies applicable for therapy of allergy must not be capable of reacting with IgE bound on sensitized mast cells and basophils, but should retain the capability to recognize sIgE⁺ B cells. Such IgE isotype-specific antibodies have been described e.g. by Chang et al. (Biotechnology 8, 122-126 (1990)), in European Patent No. EP0407392, and several U.S. patents, e.g., U.S. Pat. No. 5,449,760.

Peptides used to generate anti-IgE antibodies also suffer from the dangerous potential to induce anaphylactic antibodies. Generation of anti-IgE antibodies during active vaccination may be capable of triggering histamine release in the same way passively administered anti-IgE antibodies, if the antibodies generated during immunization bind to IgE bound to the high affinity IgE receptor or by other mechanisms.

Thus, there is a need for higher affinity non-anaphylactic antibodies that bind specifically to IgE but do not bind IgE already bound to its high affinity receptor, as well as peptides for active immunization that do not induce anaphylactic antibodies. The inventors have identified the specific epitope of IgE that provides high affinity binding of antibodies without binding to IgE on mast cells or basophils. These specific epitopes can in turn be used to generate specific peptides for active immunization to generate antibodies to IgE that only bind to the region of IgE that binds to the receptor, ensuring that the antibodies do not crosslink IgE already bound to the receptor and thus are non-anaphylactic.

### SUMMARY OF THE INVENTION

The present invention relates to novel peptide epitopes derived from the CH3 domain of IgE. These peptide epitopes are recognized by high affinity antibodies that specifically bind IgE. These novel peptides may be used for both the active immunization of a mammal by administering these peptides to generate high affinity antibodies in the mammal. The peptide epitopes may also be used in generating high affinity anti-IgE antibodies in a non-human host that specifically bind to these regions of IgE and use the resulting antibodies for the passive immunization of a of a mammal.

One peptide (epitope A, FIG. 11) of the present invention comprises the amino acid sequence:

(SEQ ID NO: 72)
Asn Pro Arg Gly Val Ser Xaa Tyr Xaa Xaa Arg Xaa.

One example of epitope A is:

(SEQ ID NO: 73)
Asn Pro Arg Gly Val Ser Ala Tyr Leu Ser Arg Pro

Another peptide (epitope B, FIG. 11) comprises the amino acid sequence:

(SEQ ID NO: 74)
Leu Pro Arg Ala Leu Xaa Arg Ser Xaa.

Examples of Epitope B include:

(SEQ ID NO: 75)
Leu Pro Arg Ala Leu Met Arg Ser Thr
(SEQ ID NO: 76)
His Pro His Leu Pro Arg Ala Leu Met Arg Ser Thr
(SEQ ID NO: 77)
Leu Pro Arg Ala Leu Met Arg Ser Thr Thr Lys Thr.

In either SEQ ID NO: 72 or SEQ ID NO: 74, Xaa may be any amino acid.

These peptides may be included in a composition comprising at least one of the peptides and a physiologically acceptable carrier, diluent, stabilizer or excipient, as well as an immunogenic carrier. The immunogenic carrier may be, e.g., BSA, KLH, tetanus toxoid, and diphtheria toxoid. The present invention also relates to polynucleotides encoding SEQ ID NOS. 72-77, vectors comprising said polynucleotides, and cells harboring said vectors.

The present invention also relates to antibodies that specifically bind to epitope A and/or epitope B. The present invention is also directed to a method of making antibodies that specifically bind to epitope A and/or epitope B.

The present invention relates to the administration of peptides comprising SEQ ID NO: 72 and/or SEQ ID NO 74 to a subject suffering from an IgE-mediated disease or condition.

The present invention relates to the administration of high affinity antibodies generated using peptides comprising SEQ ID NO: 72 and/or SEQ ID NO 74 to a mammal suffering from an IgE-mediated disease or condition. The high affinity antibody may be human, humanized, or chimeric. The antibody may be polyclonal or monoclonal. Such IgE mediated diseases or conditions include, e.g., asthma, atopic dermatitis, urticaria, allergic rhinitis and eczema.

### BRIEF DESCRIPTION OF THE FIGURES

FIG. 1 is a schematic representation of the phage vector used in antibody cloning and screening.

FIG. 2 is a schematic representation of oligonucleotides useful in generating antibody variants.

FIG. 3A depicts the comparison of the tight chains of the murine anti-IgE antibody TES-C21 and the combined human template of L16 and JK4.

FIG. 3B depicts the comparison of the heavy chains of TES-C21 and the combined human template DP88 and JH4b.

FIG. 4 presents a table of the framework residue variants having high affinity as compared to the parent TES-C21.

FIGS. 5A and B depict the ELISA titration curves for clones 4, 49, 72, 78, and 136, as compared to the parent Fab of TES-C21 and negative control (5D12).

FIG. 6 depicts an inhibition assay of clones 2C, 5A, and 51, as compared to the parent TES-C21 and a negative control antibody.

FIG. 7A depicts the sequences of clones having a combination of beneficial mutations which resulted in even greater affinity for IgE.

FIGS. 8A & 8B depict the framework sequences of the entire light chain variable region for clones 136, 1, 2, 4, 8, 13, 15, 21, 30, 31, 35, 43, 44, 53, 81, 90, and 113.

FIGS. 9A & 9B depict the framework sequences of the entire heavy chain variable region for 35 clones.

FIGS. 10 A-F depict the complete heavy and light chain sequences for clones 136, 2C, 5I, 5A, 2B, and 1136-2C.

FIG. 11 depicts the CH3 region amino acid sequence of human IgE and highlights Epitope “A” and Epitope “B”.

FIG. 12 depicts the overlapping peptides used to identify Epitope B.

FIG. 13 depicts the identification of important residues in the binding region of Epitope A.

FIG. 14 depicts the identification of important residues in the binding region of Epitope B.

FIG. 15 depicts a western blot analysis of MAb binding to mutant peptides.

FIG. 16 depicts the generation of anti-IgE antibodies in a transgenic animal expressing human IgE.

### DETAILED DESCRIPTION OF THE INVENTION

### Definitions

Terms used throughout this application are to be construed with ordinary and typical meaning to those of ordinary skill in the art. However, Applicants desire that the following terms be given the particular definition as defined below.

The phrase “substantially identical” with respect to an antibody chain polypeptide sequence may be construed as an antibody chain exhibiting at least 70%, or 80%, or 90% or 95% sequence identity to the reference polypeptide sequence. The term with respect to a nucleic acid sequence may be construed as a sequence of nucleotides exhibiting at least about 85%, or 90%, or 95% or 97% sequence identity to the reference nucleic acid sequence.

The term “identity” or “homology” shall be construed to mean the percentage of amino acid residues in the candidate sequence that are identical with the residue of a corresponding sequence to which it is compared, after aligning the sequences and introducing gaps, if necessary to achieve the maximum percent identity for the entire sequence, and not considering any conservative substitutions as part of the sequence identity. Neither N- or C-terminal extensions nor insertions shall be construed as reducing identity or homology. Methods and computer programs for the alignment are well known in the art. Sequence identity may be measured using sequence analysis software.

The term “antibody” is used in the broadest sense, and specifically covers monoclonal antibodies (including full length monoclonal antibodies), polyclonal antibodies, and multispecific antibodies (e.g., bispecific antibodies). Antibodies (Abs) and immunoglobulins (Igs) are glycoproteins having the same structural characteristics. While antibodies exhibit binding specificity to a specific target, immunoglobulins include both antibodies and other antibody-like molecules which lack target specificity. Native antibodies and immunoglobulins are usually heterotetrameric glycoproteins of about 150,000 daltons, composed of two identical light (L) chains and two identical heavy (H) chains. Each heavy chain has at one end a variable domain (V_{H}) followed by a number of constant domains. Each light chain has a variable domain at one end (V_{L}) and a constant domain at its other end. “High affinity” antibodies refers to those antibodies having a binding affinity of at least 10⁻¹⁰, preferably 10⁻¹².

As used herein, “anti-human IgE antibody” means an antibody which binds to human IgE in such a manner so as to inhibit or substantially reduce the binding of such IgE to the high affinity receptor, FcεRI.

The term “variable” in the context of variable domain of antibodies, refers to the fact that certain portions of the variable domains differ extensively in sequence among antibodies and are used in the binding and specificity of each particular antibody for its particular target. However, the variability is not evenly distributed through the variable domains of antibodies. It is concentrated in three segments called complementarity determining regions (CDRs) also known as hypervariable regions both in the light chain and the heavy chain variable domains. The more highly conserved portions of variable domains are called the framework (FR). The variable domains of native heavy and light chains each comprise four FR regions, largely a adopting a β-sheet configuration, connected by three CDRs, which form loops connecting, and in some cases forming part of, the β-sheet structure. The CDRs in each chain are held together in close proximity by the FR regions and, with the CDRs from the other chain, contribute to the formation of the target binding site of antibodies (see Kabat et al.) As used herein, numbering of immunoglobulin amino acid residues is done according to the immunoglobulin amino acid residue numbering system of Kabat et al., (Sequences of Proteins of Immunological Interest, National Institute of Health, Bethesda, Md. 1987), unless otherwise indicated.

The term “antibody fragment” refers to a portion of a full-length antibody, generally the target binding or variable region. Examples of antibody fragments include Fab, Fab′, F(ab′)₂ and Fv fragments. The phrase “functional fragment or analog” of an antibody is a compound having qualitative biological activity in common with a full-length antibody. For example, a functional fragment or analog of an anti-IgE antibody is one which can bind to an IgE immunoglobulin in such a manner so as to prevent or substantially reduce the ability of such molecule from having the ability to bind to the high affinity receptor, FcεRI. As used herein, “functional fragment” with respect to antibodies, refers to Fv, F(ab) and F(ab′)₂ fragments. An “Fv” fragment is the minimum antibody fragment which contains a complete target recognition and binding site. This region consists of a dimer of one heavy and one light chain variable domain in a tight, non-covalent association (V_{H} dimer). It is in this configuration that the three CDRs of each variable domain interact to define an target binding site on the surface of the V_{H}-V_{L} dimer. Collectively, the six CDRs confer target binding specificity to the antibody. However, even a single variable domain (or half of an Fv comprising only three CDRs specific for an target) has the ability to recognize and bind target, although at a lower affinity than the entire binding site. “Single-chain Fv” or “sFv” antibody fragments comprise the V_{H} and V_{L} domains of an antibody, wherein these domains are present in a single polypeptide chain. Generally, the Fv polypeptide further comprises a polypeptide linker between the V_{H} and V_{L} domains which enables the sFv to form the desired structure for target binding.

The Fab fragment contains the constant domain of the light chain and the first constant domain (CH1) of the heavy chain. Fab′ fragments differ from Fab fragments by the addition of a few residues at the carboxyl terminus of the heavy chain CH1 domain including one or more cysteines from the antibody hinge region. F(ab′) fragments are produced by cleavage of the disulfide bond at the hinge cysteines of the F(ab′)₂ pepsin digestion product. Additional chemical couplings of antibody fragments are known to those of ordinary skill in the art.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single targetic site. Furthermore, in contrast to conventional (polyclonal) antibody preparations which typically include different antibodies directed against different determinants (epitopes), each monoclonal antibody is directed against a single determinant on the target. In addition to their specificity, monoclonal antibodies are advantageous in that they may be synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies for use with the present invention may be isolated from phage antibody libraries using the well known techniques. The parent monoclonal antibodies to be used in accordance with the present invention may be made by the hybridoma method first described by Kohler and Milstein, Nature 256, 495 (1975), or may be made by recombinant methods.

“Humanized” forms of non-human (e.g. murine) antibodies are chimeric immunoglobulins, immunoglobulin chains or fragments thereof (such as Fv, Fab, Fab′, F(ab′)₂ or other target-binding subsequences of antibodies) which contain minimal sequence derived from non-human immunoglobulin. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody may also comprise at least a portion of an immunoglobulin constant region (Fc), typically that of a human immunoglobulin template chosen.

The terms “cell”, “cell line” and “cell culture” include progeny. It is also understood that all progeny may not be precisely identical in DNA content, due to deliberate or inadvertent mutations. Variant progeny that have the same function or biological property, as screened for in the originally transformed cell, are included. The “host cells” used in the present invention generally are prokaryotic or eukaryotic hosts.

“Transformation” of a cellular organism with DNA means introducing DNA into an organism so that the DNA is replicable, either as an extrachromosomal element or by chromosomal integration. “Transfection” of a cellular organism with DNA refers to the taking up of DNA, e.g., an expression vector, by the cell or organism whether or not any coding sequences are in fact expressed. The terms “transfected host cell” and “transformed” refer to a cell in which DNA was introduced. The cell is termed “host cell” and it may be either prokaryotic or eukaryotic. Typical prokaryotic host cells include various strains of E. coli. Typical eukaryotic host cells are mammalian, such as Chinese hamster ovary or cells of human origin. The introduced DNA sequence may be from the same species as the host cell of a different species from the host cell, or it may be a hybrid DNA sequence, containing some foreign and some homologous DNA.

The term “vector” means a DNA construct containing a DNA sequence which is operably linked to a suitable control sequence capable of effecting the expression of the DNA in a suitable host. Such control sequences include a promoter to effect transcription, an optional operator sequence to control such transcription, a sequence encoding suitable mRNA ribosome binding sites, and sequences which control the termination of transcription and translation. The vector may be a plasmid, a phage particle, or simply a potential genomic insert. Once transformed into a suitable host, the vector may replicate and function independently of the host genome, or may in some instances, integrate into the genome itself. In the present specification, “plasmid” and “vector” are sometimes used interchangeably, as the plasmid is the most commonly used form of vector. However, the invention is intended to include such other forms of vectors which serve equivalent function as and which are, or become, known in the art.

The expression “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers. DNA for a presequence or secretory leader may be operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous.

“Mammal” for purposes of treatment refers to any animal classified as a mammal, including human, domestic and farm animals, nonhuman primates, and zoo, sports, or pet animals, such as dogs, horses, cats, cows, etc.

The term “epitope tagged” when used herein in the context of a polypeptide refers to a polypeptide fused to an “epitope tag”. The epitope tag polypeptide has enough residues to provide an epitope against which an antibody can be made, yet is short enough such that it does not interfere with activity of the polypeptide. The epitope tag preferably also is fairly unique so that the antibody does not substantially cross-react with other epitopes. Suitable tag polypeptides generally have at least 6 amino acid residues and usually between about 8-50 amino acid residues (preferably between about 9-30 residues). Examples include the flu HA tag polypeptide and its antibody 12CA5 (Field et al, Mol. Cell. Biol. 8: 2159-2165 (1988))); the c-myc tag and the 8F9, 3C7, 6E10, G4, B7 and 9E10 antibodies thereagainst (Evan et al., Mol. Cell. Biol. 5(12): 3610-3616 (1985)); and the Herpes Simplex virus glycoprotein D (gD) tag and its antibody (Paborsky et al., Protein Engineering 3(6): 547-553 (1990)). In certain embodiments, the epitope tag may be an epitope of the Fc region of an IgG molecule (e.g., IgG1, IgG2, IgG3 or IgG4) that is responsible for increasing the in vivo serum half-life of the IgG molecule.

The word “label” when used herein refers to a detectable compound or composition which can be conjugated directly or indirectly to a molecule or protein, e.g., an antibody. The label may itself be detectable (e.g., radioisotope labels or fluorescent labels) or, in the case of an enzymatic label, may catalyze chemical alteration of a substrate compound or composition which is detectable.

As used herein, “solid phase” means a non-aqueous matrix to which the antibody of the present invention can adhere. Example of solid phases encompassed herein include those formed partially or entirely of glass (e.g. controlled pore glass), polysaccharides (e.g., agarose), polyacrylamides, polystyrene, polyvinyl alcohol and silicones. In certain embodiments, depending on the context, the solid phase can comprise the well of an assay plate; in others it is a purification column (e.g. an affinity chromatography column).

As used herein, the term “IgE-mediated disorder” means a condition or disease which is characterized by the overproduction and/or hypersensitivity to the immunoglobulin IgE. Specifically it would be construed to include conditions associated with anaphylactic hypersensitivity and atopic allergies, including for example: asthma, allergic rhinitis & conjunctivitis (hay fever), eczema, urticaria, atopic dermatitis, and food allergies. The serious physiological condition of anaphylactic shock caused by, e.g., bee stings, snake bites, food or medication, is also encompassed under the scope of this term.

### Generation of Antibodies

The starting or “parent” antibody may be prepared using techniques available in the art for generating such antibodies. These techniques are well known. Exemplary methods for generating the starting antibody are described in more detail in the following sections. These descriptions are possible alternatives for making or selecting a parent antibody and in no way limit the methods by which such a molecule may be generated.

The antibody's binding affinity is determined prior to generating a high affinity antibody of the present invention. Also, the antibody may be subjected to other biological activity assays, e.g., in order to evaluate effectiveness as a therapeutic. Such assays are known in the art and depend on the target and intended use for the antibody.

To screen for antibodies which bind to a particular epitope (e.g., those which block binding of IgE to its high affinity receptor), a routine cross-blocking assay such as that described in “Antibodies: A Laboratory Manual” (Cold Spring Harbor Laboratory, Ed Harlow and David Lane (1988)) can be performed. Alternatively, epitope mapping can be performed to determine where the antibody binds an epitope of interest. Optionally, the binding affinity of the antibody for a homolog of the target used to generate the antibody (where the homolog is from a different species) may be assessed using techniques known in the art. In one embodiment, the other species is a nonhuman mammal to which the antibody will be administered in preclinical studies. Accordingly, the species may be a nonhuman primate, such as rhesus, cynomolgus, baboon, chimpanzee and macaque. In other embodiments, the species may be a rodent, cat or dog, for example.

The parent antibody is altered according to the present invention so as to generate an antibody which has a higher or stronger binding affinity for the target than the parent antibody. Antibody specificity results from the unique interface that is formed between the antibody and its target; the surfaces complement each other to produce a unique fit (Jones, S. & Thornton, J. M. (1996) Proc. Natl. Acad. Sci. USA 93: 13-20). By further improving the contacts along this interface, the overall affinity can increase as a result of the lower energy cost needed to favor the association of the binding partners.

The binding surface of the antibody is generally composed of six complemetarity determining regions (CDRs) which are loops that extend out from the core. The CDRs are composed of amino acids having a sequence that is unique for binding to the specific target. To increase the affinity of an antibody for its antigen, the environment around these amino acids must become more favorable by introducing or improving various noncovalent forces, which ultimately lowers the energetics of the interaction, resulting in higher affinity.

Van der Waals forces are noncovalent interactions which occur between two electrically neutral molecules (Voet, D. & Voet, J. G. (1990) Biochemistry John Wiley and Sons, NY, N.Y.). Associations can occur between two surfaces from electrostatic interactions that arise from permanent or induced dipoles. These dipoles can exist along the ends of α-helices or near polar amino acids. By increasing the number of van der Waals forces along a binding interface, a more favorable association will result.

Introducing hydrogen bonds will also increase the specificity of an interaction between an antibody and its antigen. Common donors and acceptors involved in hydrogen bonding are nitrogen, oxygen and sulfer atoms, of which amino acids are predominantly composed (See Voet, et al., supra). Hydrogen bonds tend cross only short distances (usually 2.7 to 3.1 \acute{Å}), hence the binding partners must come within close proximity for these interactions to occur. Thus, one manner by which affinity can be improved is to bring potential donor and acceptor molecules into close contact to establish hydrogen bonds.

Finally, improving the hydrophobic interactions will also increase the favorable energetics between two binding partners. The nonpolar residues that lie close to the binding surface should be surrounded by other nonpolar residues, and thus will exist in a favorable environment. By satisfying the burial of nonpolar side chains, the energetics of the interaction are favorable for a strong binding interface.

Interactions that stabilize the protein-protein interface lower the energetic cost of maintaining these contacts and thus will increase the overall affinity. By improving the environment around individual amino acids that are near the binding interface, a more favorable environment is produced resulting in higher binding affinity. Therefore, by introducing favorable contacts and improving the interface through further complementation, the overall binding interaction between antibody and antigen will be greatly improved.

The resulting high affinity antibody preferably has a binding affinity for the target which is at least about 10 fold higher, or at least about 20 fold higher, or at least about 500 fold higher or may be 1000 to 5000 fold higher, than the binding affinity of the parent antibody for the target. The degree of enhancement in binding affinity necessary or desired will depend on the initial binding affinity of the parent antibody.

In general, the method for making high affinity antibodies from a parent antibody involves the following steps:

1. Obtaining or selecting a parent antibody which binds the target of interest, which comprises heavy and light chain variable domains. This may be done by traditional hybridoma techniques, phage-display techniques, or any other method of generating a target specific antibody.

2. Selecting a framework sequence which is close in sequence to the parent framework, preferably a human template sequence. This template may be chosen based on, e.g., its comparative overall length, the size of the CDRs, the amino acid residues located at the junction between the framework and the CDRs, overall homology, etc. The template chosen can be a mixture of more than one sequence or may be a consensus template.

3. Generating a library of clones by making random amino acid substitutions at each and every possible CDR position. One may also randomly substitute the amino acids in the human framework template that are, e.g., adjacent to the CDRs or that may affect binding or folding, with all possible amino acids, generating a library of framework substitutions. These framework substitutions can be assessed for their potential effect on target binding and antibody folding. The substitution of amino acids in the framework may be done either simultaneously or sequentially with the substitution of the amino acids in the CDRs. One method for generating the library of variants by oligonucleotide synthesis.

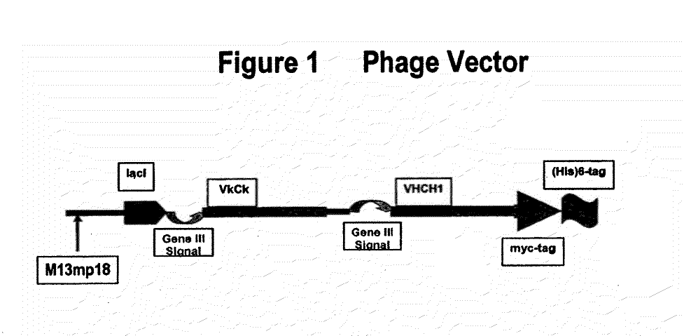

4. Constructing an expression vector comprising the heavy and/or light chain variants generated in step (3) which may comprise the formulas: FRH1-CDRH1-FRH2-CDRH2-FRH3-CDRH3-FRH4(I) and FRL1-CDRL1-FRL2-CDRL2-FRL3-CDRL3-FRL4 (II), wherein FRL1, FRL2, FRL3, FRL4, FRH1, FRH2. FRH3 and FRH4 represent the variants of the framework template light chain and heavy chain sequences chosen in step 3 and the CDRs represent the variant CDRs of the parent antibody CDRs. An example of a vector containing such light and heavy chain sequences is depicted in FIG. 1.

5. Screening the library of clones against the specific target and those clones that bind the target are screened for improved binding affinity. Those clones that bind with greater affinity than the parent molecule may be selected. The optimal high affinity candidate will have the greatest binding affinity possible compared to the parent antibody, preferably greater then 20 fold, 100 fold, 1000 fold or 5000 fold. If the chosen variant contains certain amino acids that are undesirable, such as a glycosylation site that has been introduced or a potentially immunogenic site, those amino acids may be replaced with more beneficial amino acid residues and the binding affinity reassessed.

One may also use this method to generate high affinity antibodies from a fully human parent antibody by randomly substituting only the CDR regions, leaving the human framework intact.

Due to improved high throughput screening techniques and vectors such as the one depicted in FIG. 1, an artisan can rapidly and efficiently screen a comprehensive library of substitutions at all sites in a given CDR and/or framework region. By randomly substituting all amino acids at all positions simultaneously, one is able to screen possible combinations that significantly increase affinity that would not have been anticipated or identified by individual substitution due to, e.g., synergy.

### Parent Antibody Preparation

Target Preparation

Soluble targets or fragments thereof can be used as immunogens for generating antibodies. The antibody is directed against the target of interest. Preferably, the target is a biologically important polypeptide and administration of the antibody to a mammal suffering from a disease or disorder can result in a therapeutic benefit in that mammal. However, antibodies may be directed against nonpolypeptide targets. Where the target is a polypeptide, it may be a transmembrane molecule (e.g. receptor) or ligand such as a growth factor. One target of the present invention is IgE. Whole cells may be used as the immunogen for making antibodies. The target may be produced recombinantly or made using synthetic methods. The target may also be isolated from a natural source.

Antigens used in producing antibodies of the invention may include polypeptides and polypeptide fragments of the invention, including epitope A and/or B. A polypeptide used to immunize an animal can be obtained by standard recombinant, chemical synthetic, or purification methods. As is well known in the art, in order to increase immunogenicity, an antigen can be conjugated to a carrier protein. Commonly used carriers include, but are not limited to, keyhole limpet hemocyanin (KLH), thyroglobulin, bovine serum albumin (BSA), and tetanus toxoid. The coupled peptide is then used to immunize an animal (e.g., a mouse, a rat, or a rabbit). In addition to such carriers, well known adjuvants can be administered with the antigen to facilitate induction of a strong immune response.

Polyclonal Antibodies

Polyclonal antibodies are usually generated in non-human mammals by multiple subcutaneous (sc) or intraperitoneal (ip) injections of the relevant target in combination with an adjuvant. Numerous agents capable of eliciting an immunological response are well known in the art.

Animals are immunized against the target, immunogenic conjugates, or derivatives by combining the protein or conjugate (for rabbits or mice, respectively) with Freund's complete adjuvant and injecting the solution intradermally. One month later the animals are boosted with ⅕ to 1/10 the original amount of peptide or conjugate in Freund's incomplete adjuvant by subcutaneous injection at multiple sites. Seven to 14 days later the animals are bled and the serum is assayed for antibody titer. Animals are boosted until the titer plateaus.

The mammalian antibody selected will normally have a sufficiently strong binding affinity for the target. For example, the antibody may bind the human anti-IgE target with a binding affinity (Kd) value of about 1×10⁻⁸ M. Antibody affinities may be determined by saturation binding; enzyme-linked immunoabsorbant assay (ELISA); and competition assays (e.g., radioimmunoassays).

To screen for antibodies that bind the target of interest, a routine cross-linking assay such as that described in Antibodies, A Laboratory Manual, Cold Spring Harbor Laboratory, Ed Harlow and David Lane (1988) can be performed. Alternatively, epitope mapping, e.g., as described in Champe, et al. J. Biol. Chem. 270: 1388-1394 (1995), can be performed to determine binding.

Monoclonal Antibodies

Monoclonal antibodies are antibodies which recognize a single antigenic site. Their uniform specificity makes monoclonal antibodies much more useful than polyclonal antibodies, which usually contain antibodies that recognize a variety of different antigenic sites. Monoclonal antibodies may be made using the hybridoma method first described by Kohler et al., Nature, 256: 495 (1975), or may be made by recombinant DNA methods.

In the hybridoma method, a mouse or other appropriate host animal, such as a rodent, is immunized as hereinabove described to elicit lymphocytes that produce or are capable of producing antibodies that will specifically bind to the protein used for immunization. Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principals and Practice, pp. 590-103 (Academic Press, 1986)).

The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), substances which prevent the growth of HGPRT-deficient cells. Preferred myeloma cells are those that fuse efficiently, support stable high-level production of antibody by the selected antibody-producing cells, and are sensitive to a medium such as HAT medium. Human myeloma and mouse-human heteromyeloma cell lines have been described for the production of human monoclonal antibodies (Kozbar, J. Immunol. 133: 3001 (1984); Brodeur et al., Monoclonal Antibody Production Techniques and Applications, pp. 51-63 (Marcel Dekker, Inc., New York, 1987)).

After hybridoma cells are identified that produce antibodies of the desired specificity, affinity, and/or activity, the clones may be subcloned by limiting dilution procedures and grown by standard methods (Goding, Monoclonal Antibodies: Principals and Practice, pp. 59-103, Academic Press, 1986)). Suitable culture media for this purpose include. The monoclonal antibodies secreted by the subclones are suitably separated from the culture medium by conventional immunoglobulin purification procedures such as, for example, protein A-Sepharose, hydroxylapatite chromatography, gel electrophoresis, dialysis, or affinity chromatography.

DNA encoding the monoclonal antibodies is readily isolated and sequenced using conventional procedures (e.g., by using oligonucleotide probes that are capable of binding specifically to genes encoding the heavy and light chains of the monoclonal antibodies). The hybridoma cells serve as a source of such DNA. Once isolated, the DNA may be placed into expression vectors, which are then transferred into host cells such as E. coli cells, NSO cells, Chinese hamster ovary (CHO) cells, or myeloma cells to obtain the synthesis of monoclonal antibodies in the recombinant host cells. The DNA also may be modified, for example, by substituting the coding sequence for human heavy- and light-chain constant domains in place of the homologous murine sequences (U.S. Pat. No. 4,816,567; Morrison et al., Proc. Natl. Acad. Sci. USA 81: 6851 (1984)), or by covalently joining to the immunoglobulin polypeptide.

Humanized Antibodies

Humanization is a technique for making a chimeric antibody wherein substantially less than an intact human variable domain has been substituted by the corresponding sequence from a non-human species. A humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers (Jones et al, Nature 321: 522-525 (1986); Riechman et al., Nature 332: 323-327 (1988); Verhoeyens et al., Science 239: 1534-1536 (1988)), by substituting non-human CDR's or CDR sequences for the corresponding sequences in a human antibody (See, e.g., U.S. Pat. No. 4,816,567). As practiced in the present invention, the humanized antibody may have some CDR residues and some FR residues substituted by residues from analogous sites in murine antibodies.

The choice of human variable domains, both light and heavy, to be used in making the humanized antibodies is very important to reduce antigenicity. According to the so-called “best fit” method, the sequence of the variable domain of a non-human antibody is compared with the library of known human variable-domain sequences. The human sequence which is closest to that of the non-human parent antibody is then accepted as the human framework for the humanized antibody (Sims et al., J. Immunol. 151: 2296 (1993); Chothia et al., J. Mol. Biol. 196: 901 (1987)). Another method uses a particular framework derived from the consensus sequence of all human antibodies of a particular subgroup of light or heavy chains. The same framework may be used for several different humanized antibodies (Carter et al., Proc. Natl. Acad. Sci. USA, 89: 4285 (1992); Presta et al., J. Immunol. 151: 2623 (1993)).

Antibody Fragments

Various techniques have been developed for the production of antibody fragments. Traditionally, these fragments were derived via proteolytic digestion of intact antibodies (see, e.g., Morimoto et al., Journal of Biochemical and Biophysical Methods 24: 107-117 (1992) and Brennan et al., Science 229: 81 (1985)). However, these fragments can now be produced directly by recombinant host cells. For example, the antibody fragments can be isolated from an antibody phage library. Alternatively, F(ab′)₂SH fragments can be directly recovered from E. coli and chemically coupled to form F(ab′)₂ fragments (Carter et al., Bio/Technology 10: 163-167 (1992)). According to another approach, F(ab′)₂ fragments can be isolated directly from recombinant host cell culture. Other techniques for the production of antibody fragments will be apparent to the skilled practitioner. In other embodiments, the antibody of choice is a single chain Fv fragment (scFv). (PCT patent application WO 93/16185).

### Preparation of High Affinity Antibodies

Once the parent antibody has been identified and isolated, one or more amino acid residues may be altered in one or more of the variable regions of the parent antibody. Alternatively, or in addition, one or more substitutions of framework residues may be introduced in the parent antibody where these result in an improvement in the binding affinity of the antibody, for example, for human IgE. Examples of framework region residues to modify include those which non-covalently bind target directly (Amit et al. Science 233: 747-753 (1986)); interact with/effect the conformation of CDR (Chothia et al. J. Mol. Biol. 196: 901-917 (1987)); and/or participate in the VL-VH interface (EP 239 400 B1). In certain embodiments, modification of one or more of such framework region residues results in an enhancement of the binding affinity of the antibody for the target of interest.

Modifications in the antibodies' biological properties may be accomplished by selecting substitutions that differ significantly in their effect on maintaining, e.g., (a) the structure of the polypeptide backbone in the area of the substitution, for example, as a sheet or helical conformation; (b) the charge or hydrophobicity of the molecule at the target site, or (c) the bulk of the side chain. Non-conservative substitutions will entail exchanging a member of one of these classes for another class.

Nucleic acid molecules encoding amino acid sequence variants are prepared by a variety of methods known in the art. These methods include, but are not limited to, oligonucleotide-mediated (or site-directed) mutagenesis, PCR mutagenesis, and cassette mutagenesis of an earlier prepared variant or a non-variant version of the species-dependent antibody. The preferred method for generating variants is an oligonucleotide-mediated synthesis. In certain embodiments, the antibody variant will only have a single hypervariable region residue substituted, e.g. from about two to about fifteen hypervariable region substitutions.

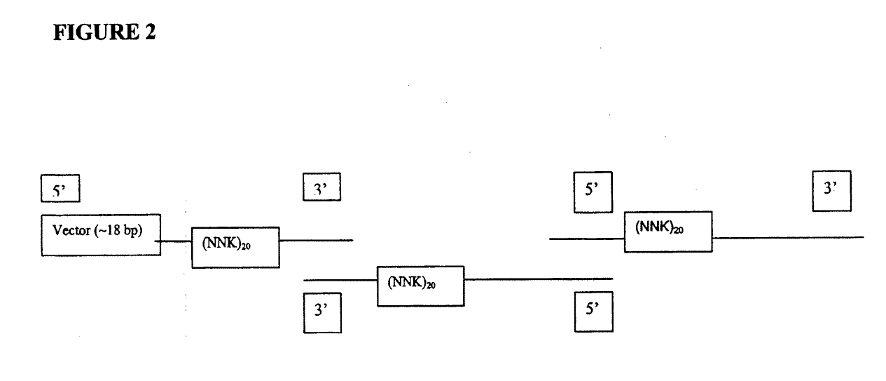

One method for generating the library of variants is by oligonucleotide mediated synthesis according to the scheme depicted in FIG. 2. Three oligonucleotides of approximately 100 nucleotides each may be synthesized spanning the entire light chain or heavy chain variable region. Each oligonucleotide may comprise: (1) a 60 amino acid stretch generated by the triplet (NNK)₂₀ where N is any nucleotide and K is G or T, and (2) an approximately 15-30 nucleotide overlap with either the next oligo or with the vector sequence at each end. Upon annealing of these three oligonucleotides in a PCR reaction, the polymerase will fill in the opposite strand generating a complete double stranded heavy chain or light chain variable region sequence. The number of triplets may be adjusted to any length of repeats and their position within the oligonucleotide may be chosen so as to only substitute amino acds in a given CDR or framework region. By using (NNK), all twenty amino acids are possible at each position in the encoded variants. The overlapping sequence of 5-10 amino acids (15-30 nucloetides) will not be subtituted, but this may be chosen to fall within the stacking regions of the framework, or may substituted by a separate or subsequent round of synthesis. Methods for synthesizing oligonucleotides are well known in the art and are also commercially available. Methods for generating the antibody variants from these oligonucleotides are also well known in the art, e.g., PCR.

The library of heavy and light chain variants, differing at random positions in their sequence, can be constructed in any expression vector, such as a bacteriophage, specifically the vector of FIG. 1, each of which contains DNA encoding a particular heavy and light chain variant.

Following production of the antibody variants, the biological activity of variant relative to the parent antibody is determined. As noted above, this involves determining the binding affinity of the variant for the target. Numerous high-throughput methods exist for rapidly screen antibody variants for their ability to bind the target of interest.

One or more of the antibody variants selected from this initial screen may then be screened for enhanced binding affinity relative to the parent antibody. One common method for determining binding affinity is by assessing the association and dissociation rate constants using a BIAcore™ surface plasmon resonance system (BIAcore, Inc.). A biosensor chip is activated for covalent coupling of the target according to the manufacturer's (BIAcore) instructions. The target is then diluted and injected over the chip to obtain a signal in response units (RU) of immobilized material. Since the signal in RU is proportional to the mass of immobilized material, this represents a range of immobilized target densities on the matrix. Dissociation data are fit to a one-site model to obtain k_{off}+/−s.d. (standard deviation of measurements). Pseudo-first order rate constant (kₛ) are calculated for each association curve, and plotted as a function of protein concentration to obtain kₒₙ+/−s.e. (standard error of fit). Equilibrium dissociation constants for binding, K_{D}'s, are calculated from SPR measurements as k_{off}/kₒₙ. Since the equilibrium dissociation constant, K_{D}, is inversely proportional to k_{off}, an estimate of affinity improvement can be made assuming the association rate (kₒₙ) is a constant for all variants.

The resulting candidate(s) with high affinity may optionally be subjected to one or more further biological activity assays to confirm that the antibody variant(s) with enhanced binding affinity still retain the desired therapeutic attributes. For example, in the case of an anti-IgE antibody, one may screen for those that block binding of IgE to its receptor and inhibit the release of histamine. The optimal antibody variant retains the ability to bind the target with a binding affinity significantly higher than the parent antibody.

The antibody variant(s) so selected may be subjected to further modifications oftentimes depending upon the intended use of the antibody. Such modifications may involve further alteration of the amino acid sequence, fusion to heterologous polypeptide(s) and/or covalent modifications such as those elaborated below. For example, any cysteine residues not involved in maintaining the proper conformation of the antibody variant may be substituted, generally with serine, to improve the oxidative stability of the molecule and prevent aberrant cross linking. Conversely, (a) cysteine bond(s) may be added to the antibody to improve its stability (particularly where the antibody is an antibody fragment such as an Fv fragment).

### Vectors

The invention also provides isolated nucleic acid encoding an antibody variant as disclosed herein, vectors and host cells comprising the nucleic acid, and recombinant techniques for the production of the antibody variant. For recombinant production of the antibody variant, the nucleic acid encoding it is isolated and inserted into a replicable vector for further cloning (amplification of the DNA) or for expression. DNA encoding the antibody variant is readily isolated and sequenced using conventional procedures (e.g., by using oligonucleotide probes that are capable of binding specifically to genes encoding the heavy and light chains of the antibody variant).

Many vectors are available. The vector components generally include, but are not limited to, one or more of the following: a signal sequence, an origin of replication, one or more marker genes, an enhancer element, a promoter, and a transcription termination sequence.

The phage expression vector depicted in FIG. 1 is comprised of a commonly used M13 vector and M13's own gene III viral secretion signal for rapid secretion and screening variant Fabs for proper binding specificity and minimal affinity criteria. This vector does not use the entire gene III sequence, so there is no display on the surface of the bacterial cell, but rather the Fabs are secreted into the periplasmic space. Alternatively, the Fabs could be expressed in the cytoplasm and isolated. The heavy and light chains each have their own viral secretion signal, but are dependently expressed from a single strong inducible promoter.

The vector in FIG. 1 also provides a His tag and a myc tag for easy purification, as well as detection. A skilled artisan would recognize that the Fabs could be independently expressed from separate promoters or that the secretion signal need not be the viral sequence chosen, but could be a prokaryotic or eukaryotic signal sequence suitable for the secretion of the antibody fragments from the chosen host cell. It should also be recognized that the heavy and light chains may reside on different vectors.

A. Signal Sequence Component

The antibody variant of this invention may be produced recombinantly. The variant may also be expressed as a fusion polypeptide fused with a heterologous polypeptide, which is preferably a signal sequence or other polypeptide having a specific cleavage site at the N-terminus of the mature protein or polypeptide. The heterologous signal sequence selected preferably is one that is recognized and processed (i.e., cleaved by signal peptidase) by the host cell. For prokaryotic host cells that do not recognize and process the native antibody signal sequence, the signal sequence may be substituted by a prokaryotic signal sequence selected, for example, from the group of the alkaline phosphatase, penicillinase, Ipp, or heat-stable enterotoxin II leaders. Or in the case of the vector of FIG. 1, the signal sequence chosen was a viral signal sequence from gene III. For yeast secretion the native signal sequence may be substituted by, e.g., the yeast invertase leader, α-factor leader (including Saccharomyces and Kluyveromyces α-factor leaders), or acid phosphatase leader, the C. albicans glucoamylase leader, or a signal described in e.g., WO 90/13646. In mammalian cell expression, mammalian signal sequences as well as viral secretory leaders, for example, the herpes simplex gD signal, are available. The DNA for such precursor region is ligated in reading frame to DNA encoding the antibody variant.

B. Origin of Replication Component

Vectors usually contain a nucleic acid sequence that enables the vector to replicate in one or more selected host cells. Generally, this sequence is one that enables the vector to replicate independently of the host chromosomal DNA, and includes origins of replication or autonomously replicating sequences. Such sequences are well known for a variety of bacteria, yeast, and viruses. The origin of replication from the plasmid pBR322 is suitable for most Gram-negative bacteria, the 2μ plasmid origin is suitable for yeast, and various viral origins (SV40, polyoma, adenovirus, VSV or BPV) are useful for vectors in mammalian cells. Generally, the origin of replication component is not needed for mammalian expression vectors (the SV40 origin may typically be used only because it contains the early promoter).

C. Selection Gene Component

Vectors may contain a selection gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli.

One example of a selection scheme utilizes a drug to arrest growth of a host cell. Those cells that are successfully transformed with a heterologous gene produce a protein conferring drug resistance and thus survive the selection regimen. Examples of such dominant selection use the drugs neomycin, mycophenolic acid and hygromycin.

Another example of suitable selectable markers for mammalian cells are those that enable the identification of cells competent to take up the antibody nucleic acid, such as DHFR, thymidine kinase, metallothionein-I and -II, preferably primate metallothionein genes, adenosine deaminase, ornithine decarboxylase, etc.

For example, cells transformed with the DHFR selection gene are first identified by culturing all of the transformants in a culture medium that contain methotrexate (Mtx), a competitive antagonist of DHFR. An appropriate host cell when wild-type DHFR is employed is the Chinese hamster ovary (CHO) cell line deficient in DHFR activity.

Alternatively, host cells (particularly wild-type hosts that contain endogenous DHFR) transformed or co-transformed with DNA sequences encoding antibody, wild-type DHFR protein, and another selectable marker such as aminoglycoside 3′-phosphotransferase (APH) can be selected by cell growth in medium containing a selection agent for the selectable marker such as an aminoglycosidic antibiotic, e.g., kanamycin, neomycin, or G418. (U.S. Pat. No. 4,965,199).

A suitable selection gene for use in yeast is the trp1 gene present in the yeast plasmid Yrp7 (Stinchcomb et al., Nature 282: 39 (1979)). The trp1 gene provides a selection marker for a variant strain of yeast lacking the ability to grow in typtophan, for example, ATCC No. 44076 or PEP4-1. Jones, Genetics 85: 12 (1977). The presence of the trp1 lesion in the yeast host cell genome then provides an effective environment for detecting transformation by growth in the absence of tryptophan. Similarly, Leu2-deficient yeast strains (ATCC 20,622 or 38,626) are complemented by known plasmids bearing the Leu2 gene.

D. Promoter Component

Expression and cloning vectors usually contain a promoter that is recognized by the host organism and is operably linked to the antibody nucleic acid. Promoters suitable for use with prokaryotic hosts include the phoA promoter, β-lactamase and lactose promoter systems, alkaline phosphatase, a tryptophan (trp) promoter system, and hybrid promoters such as the tac promoter. However, other known bacterial promoters are suitable. Promoters for use in bacterial systems may also contain a Shine-Dalgarno (S.D.) sequence operably linked to the DNA encoding the antibody.

Promoter sequences are known for eukaryotes. Virtually all eukaryotic genes have an AT-rich region located approximately 25 to 30 bases upstream from the site where transcription is initiated. Another sequence found 70 to 80 bases upstream from the start of transcription of many genes is a CNCAAT region where N may be any nucleotide. At the 3′ end of most eukaryotic genes is an AATAAA sequence that may be the signal for addition of the poly A tail to the 3′ end of the coding sequence. All of these sequences are suitably inserted into eukaryotic expression vectors.

Examples of suitable promotor sequences for use with yeast hosts include the promoters for 3-phosphoglycerate kinase or other glycolytic enzymes, such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase.

Other yeast promoters, which are inducible promoters having the additional advantage of transcription controlled by growth conditions, are the promoter regions for alcohol dehydrogenase 2, isocytochrome C, acid phosphatase, degradative enzymes associated with nitrogen metabolism, metallothionein, glyceraldehyde-3-phosphate dehydrogenase, and enzymes responsible for maltose and galactose utilization. Suitable vectors and promoters for use in yeast expression are further described in EP 73,657. Yeast enhancers also are advantageously used with yeast promoters.

Antibody transcription from vectors in mammalian host cells is controlled, for example, by promoters obtained from the genomes of viruses such as polyoma virus, fowlpox virus, adenovirus (such as Adenovirus 2), bovine papilloma virus, avian sarcoma virus, cytomegalovirus, a retrovirus, hepatitis-B virus and most preferably Simian virus 40 (SV40), from heterologous mammalian promoters, e.g., the actin promoter or an immunoglobulin promoter, from heat-shock promoters—provided such promoters are compatible with the host cell systems.

The early and late promoters of the SV40 virus are conveniently obtained as an SV40 restriction fragment that also contains the SV40 viral origin of replication. The immediate early promoter of the human cytomegalovirus is conveniently obtained as a HindIII E restriction fragment. A system for expressing DNA in mammalian hosts using the bovine papilloma virus as a vector is disclosed in U.S. Pat. No. 4,419,446. A modification of this system is described in U.S. Pat. No. 4,601,978. Alternatively, human β-interferon cDNA has been expressed in mouse cells under the control of a thymidine kinase promoter from herpes simplex virus. Alternatively, the rous sarcoma virus long terminal repeat can be used as the promoter.

E. Enhancer Element Component

Transcription of a DNA encoding the antibody of this invention by higher eukaryotes is often increased by inserting an enhancer sequence into the vector. Many enhancer sequences are now known from mammalian genes (globin, elastase, albumin, α-fetoprotein, and insulin). Typically, however, one will use an enhancer from a eukaryotic cell virus. Examples include the SV40 enhancer on the late side of the replication origin (bp 100-270), the cytomegalovirus early promoter enhancer, the polyoma enhancer on the late side of the replication origin, and adenovirus enhancers. See also Yaniv, Nature 297: 17-18 (1982) on enhancing elements for activation of eukaryotic promoters. The enhancer may be spliced into the vector at a position 5′ or 3′ to the antibody-encoding sequence, but is preferably located at a site 5′ from the promoter.

F. Transcription Termination Component

Expression vectors used in eukaryotic host cells (yeast, fungi, insect, plant, animal, human, or nucleated cells from other multicellular organisms) may also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the mRNA encoding the antibody. One useful transcription termination component is the bovine growth hormone polyadenylation region. See e.g., WO94/11026.

### Selection and Transformation of Host Cells

Suitable host cells for cloning or expressing the DNA in the vectors herein are prokaryotic, yeast, or higher eukaryotic cells. Suitable prokaryotes for this purpose include both Gram-negative and Gram-positive organisms, for example, Enterobacteria such as E. coli, Enterobacter, Erwinia, Klebsiella, Proteus, Salmonella, Serratia, and Shigella, as well as Bacilli, Pseudomonas, and Streptomyces. One preferred E. coli cloning host is E. coli 294 (ATCC 31,446), although other strains such as E. coli B, E. coli X1776 (ATCC 31,537), and E. coli W3110 (ATCC 27,325) are suitable. These examples are illustrative rather than limiting.

In addition to prokaryotes, eukaryotic microbes such as filamentous fungi or yeast are suitable cloning or expression hosts for antibody-encoding vectors. Saccharomyces cerevisiae is the most commonly used among lower eukaryotic host microorganisms. However, a number of other genera, species, and strains are commonly available and useful herein, such as Schizosaccharomyces pombe; Kluyveromyces; Candida; Trichoderma; Neurospora crassa; and filamentous fungi such as e.g., Neurospora, Penicillium, Tolypocladium, and Aspergillus hosts, such as A. nidulans and A. niger.

Suitable host cells for the expression of glycosylated antibodies are derived from multicellular organisms. In principal, any higher eukaryotic cell culture is workable, whether from vertebrate or invertebrate culture. Examples of invertebrate cells include plant and insect cells, Luckow et al., Bio/Technology 6, 47-55 (1988); Miller et al., Genetic Engineering, Setlow et al. eds. Vol. 8, pp. 277-279 (Plenam publishing 1986); Mseda et al., Nature 315, 592-594 (1985). Numerous baculoviral strains and variants and corresponding permissive insect host cells from hosts such as Spodoptera frugiperda (caterpillar), Aedes (mosquito), Drosophila melanogaster (fruitfly), and Bombyx mori have been identified. A variety of viral strains for transfection are publicly available, e.g., the L-1 variant of Autographa californica NPV and the Bm-5 strain of Bombyx mori NPV, and such viruses may be used as the virus herein according to the present invention, particularly for transfection of Spodoptera frugiperda cells. Moreover, plant cells cultures of cotton, corn, potato, soybean, petunia, tomato, and tobacco and also be utilized as hosts.

Vertebrate cells and propagation of vertebrate cells, in culture (tissue culture) has become routine. See Tissue Culture, Academic Press, Kruse and Patterson, eds. (1973). Examples of useful mammalian host cell lines are monkey kidney; human embryonic kidney line; baby hamster kidney cells; Chinese hamster ovary cells/-DHFR (CHO, Urlaub et al., Proc. Natl. Acad. Sci. USA 77: 4216 (1980)); mouse sertoli cells; human cervical carcinoma cells (HELA); canine kidney cells; human lung cells; human liver cells; mouse mammary tumor; and NSO cells.

Host cells are transformed with the above-described vectors for antibody production and cultured in conventional nutrient media modified as appropriate for inducing promoters, selecting transformants, or amplifying the genes encoding the desired sequences.

The host cells used to produce the antibody variant of this invention may be cultured in a variety of media. Commercially available media such as Ham's F10 (Sigma), Minimal Essential Medium (MEM, Sigma), RPMI-1640 (Sigma), and Dulbecco's Modified Eagle's Medium (DMEM, Sigma) are suitable for culturing host cells. In addition, any of the media described in Ham et al., Meth. Enzymol. 58: 44 (1979), Barnes et al., Anal. Biochem. 102: 255 (1980), U.S. Pat. No. 4,767,704; 4,657,866; 4,560,655; 5,122,469; 5,712,163; or 6,048,728 may be used as culture media for the host cells. Any of these media may be supplemented as necessary with hormones and/or other growth factors (such as insulin, transferrin, or epidermal growth factor), salts (such as X-chlorides, where X is sodium, calcium, magnesium; and phosphates), buffers (such as HEPES), nucleotides (such as adenosine and thymidine), antibiotics (such as GENTAMYCIN™ drug), trace-elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

### Antibody Purification

When using recombinant techniques, the antibody variant can be produced intracellularly, in the periplasmic space, or directly secreted into the medium. If the antibody variant is produced intracellularly, as a first step, the particulate debris, either host cells or lysed fragments, may be removed, for example, by centrifugation or ultrafiltration. Carter et al., Bio/Technology 10: 163-167 (1992) describe a procedure for isolating antibodies which are secreted to the periplasmic space of E. coli. Briefly, cell paste is thawed in the presence of sodium acetate (pH 3.5), EDTA, and phenylmethylsulfonylfluoride (PMSF) over about 30 minutes. Cell debris can be removed by centrifugation. Where the antibody variant is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The antibody composition prepared from the cells can be purified using, for example, hydroxylapatite chromatography, gel elecrophoresis, dialysis, and affinity chromatography, with affinity chromatography being the preferred purification technique. The suitability of protein A as an affinity ligand depends on the species and isotype of any immunoglobulin Fc domain that is present in the antibody variant. Protein A can be used to purify antibodies that are based on human IgG1, IgG2 or IgG4 heavy chains (Lindmark et al., J. Immunol. Meth. 62: 1-13 (1983)). Protein G is recommended for all mouse isotypes and for human IgG3 (Guss et al., EMBO J. 5: 1567-1575 (1986)). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody variant comprises a CH3 domain, the Bakerbond ABXTM resin (J. T. Baker, Phillipsburg, N.J.) is useful for purification. Other techniques for protein purification such as fractionation on an ion-exchange column, ethanol precipitation, Reverse Phase HPLC, chromatography on silica, chromatography on heparin SEPHAROSE™ chromatography on an anion or cation exchange resin (such as a polyaspartic acid column), chromatofocusing, SOS-PAGE, and ammonium sulfate precipitation are also available depending on the antibody variant to be recovered.

Following any preliminary purification step(s), the mixture comprising the antibody variant of interest and contaminants may be subjected to low pH hydrophobic interaction chromatography using an elution buffer at a pH between about 2.5-4.5, preferably performed at low salt concentrations (e.g., from about 0-0.25M salt).

### Pharmaceutical Formulations

Therapeutic formulations of the polypeptide or antibody may be prepared for storage as lyophilized formulations or aqueous solutions by mixing the polypeptide having the desired degree of purity with optional “pharmaceutically-acceptable” carriers, excipients or stabilizers typically employed in the art (all of which are termed “excipients”). For example, buffering agents, stabilizing agents, preservatives, isotonifiers, non-ionic detergents, antioxidants and other miscellaneous additives. (See Remington's Pharmaceutical Sciences, 16th edition, A. Osol, Ed. (1980)). Such additives must be nontoxic to the recipients at the dosages and concentrations employed.

Buffering agents help to maintain the pH in the range which approximates physiological conditions. They are preferably present at concentration ranging from about 2 mM to about 50 mM. Suitable buffering agents for use with the present invention include both organic and inorganic acids and salts thereof such as citrate buffers (e.g., monosodium citrate-disodium citrate mixture, citric acid-trisodium citrate mixture, citric acid-monosodium citrate mixture, etc.), succinate buffers (e.g., succinic acid-monosodium succinate mixture, succinic acid-sodium hydroxide mixture, succinic acid-disodium succinate mixture, etc.), tartrate buffers (e.g., tartaric acid-sodium tartrate mixture, tartaric acid-potassium tartrate mixture, tartaric acid-sodium hydroxide mixture, etc.), fumarate buffers (e.g., fumaric acid-monosodium fumarate mixture, etc.), fumarate buffers (e.g., fumaric acid-monosodium fumarate mixture, fumaric acid-disodium fumarate mixture, monosodium fumarate-disodium fumarate mixture, etc.), gluconate buffers (e.g., gluconic acid-sodium glyconate mixture, gluconic acid-sodium hydroxide mixture, gluconic acid-potassium glyuconate mixture, etc.), oxalate buffer (e.g., oxalic acid-sodium oxalate mixture, oxalic acid-sodium hydroxide mixture, oxalic acid-potassium oxalate mixture, etc.), lactate buffers (e.g., lactic acid-sodium lactate mixture, lactic acid-sodium hydroxide mixture, lactic acid-potassium lactate mixture, etc.) and acetate buffers (e.g., acetic acid-sodium acetate mixture, acetic acid-sodium hydroxide mixture, etc.). Additionally, there may be mentioned phosphate buffers, histidine buffers and trimethylamine salts such as Tris.

Preservatives may be added to retard microbial growth, and may be added in amounts ranging from 0.2%-1% (w/v). Suitable preservatives for use with the present invention include phenol, benzyl alcohol, meta-cresol, methyl paraben, propyl paraben, octadecyldimethylbenzyl ammonium chloride, benzalconium halides (e.g., chloride, bromide, iodide), hexamethonium chloride, alkyl parabens such as methyl or propyl paraben, catechol, resorcinol, cyclohexanol, and 3-pentanol.

Isotonicifiers sometimes known as “stabilizers” may be added to ensure isotonicity of liquid compositions of the present invention and include polhydric sugar alcohols, preferably trihydric or higher sugar alcohols, such as glycerin, erythritol, arabitol, xylitol, sorbitol and mannitol.

Stabilizers refer to a broad category of excipients which can range in function from a bulking agent to an additive which solubilizes the therapeutic agent or helps to prevent denaturation or adherence to the container wall. Typical stabilizers can be polyhydric sugar alcohols (enumerated above); amino acids such as arginine, lysine, glycine, glutamine, asparagine, histidine, alanine, ornithine, L-leucine, 2-phenylalanine, glutamic acid, threonine, etc., organic sugars or sugar alcohols, such as lactose, trehalose, stachyose, mannitol, sorbitol, xylitol, ribitol, myoinisitol, galactitol, glycerol and the like, including cyclitols such as inositol; polyethylene glycol; amino acid polymers; sulfur containing reducing agents, such as urea, glutathione, thioctic acid, sodium thioglycolate, thioglycerol, α-monothioglycerol and sodium thio sulfate; low molecular weight polypeptides (i.e. <10 residues); proteins such as human serum albumin, bovine serum albumin, gelatin or immunoglobulins; hydrophylic polymers, such as polyvinylpyrrolidone monosaccharides, such as xylose, mannose, fructose, glucose; disaccharides such as lactose, maltose, sucrose and trisaccacharides such as raffinose; polysaccharides such as dextran. Stabilizers may be present in the range from 0.1 to 10,000 weights per part of weight active protein.

Non-ionic surfactants or detergents (also known as “wetting agents”) may be added to help solubilize the therapeutic agent as well as to protect the therapeutic protein against agitation-induced aggregation, which also permits the formulation to be exposed to shear surface stressed without causing denaturation of the protein. Suitable non-ionic surfactants include polysorbates (20, 80, etc.), polyoxamers (184, 188 etc.), Pluronic® polyols, polyoxyethylene sorbitan monoethers (TWEEN®-20, TWEEN®-80, etc.). Non-ionic surfactants may be present in a range of about 0.05 mg/ml to about 1.0 mg/ml, preferably about 0.07 mg/ml to about 0.2 mg/ml.

Additional miscellaneous excipients include bulking agents, (e.g. starch), chelating agents (e.g. EDTA), antioxidants (e.g., ascorbic acid, methionine, vitamin E), and co-solvents. The formulation herein may also contain more than one active compound as necessary for the particular indication being treated, preferably those with complementary activities that do not adversely affect each other. For example, it may be desirable to further provide an immunosuppressive agent. Such molecules are suitably present in combination in amounts that are effective for the purpose intended. The active ingredients may also be entrapped in microcapsule prepared, for example, by coascervation techniques or by interfacial polymerization, for example, hydroxymethylcellulose or gelatin-microcapsule and poly-(methylmethacylate) microcapsule, respectively, in colloidal drug delivery systems (for example, liposomes, albumin micropheres, microemulsions, nano-particles and nanocapsules) or in macroemulsions. Such techniques are disclosed in Remington's Pharmaceutical Sciences, 16th edition, A. Osal, Ed. (1980).

The formulations to be used for in vivo administration must be sterile. This is readily accomplished, for example, by filtration through sterile filtration membranes. Sustained-release preparations may be prepared. Suitable examples of sustained-release preparations include semi-permeable matrices of solid hydrophobic polymers containing the antibody variant, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated antibodies remain in the body for a long time, they may denature or aggregate as a result of exposure to moisture at 37° C. resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

The amount of therapeutic polypeptide, antibody or fragment thereof which will be effective in the treatment of a particular disorder or condition will depend on the nature of the disorder or condition, and can be determined by standard clinical techniques. Where possible, it is desirable to determine the dose-response curve and the pharmaceutical compositions of the invention first in vitro, and then in useful animal model systems prior to testing in humans.

In a preferred embodiment, an aqueous solution of therapeutic polypeptide, antibody or fragment thereof is administered by subcutaneous injection. Each dose may range from about 0.5 μg to about 50 μg per kilogram of body weight, or more preferably, from about 3 μg to about 30 μg per kilogram body weight.

The dosing schedule for subcutaneous administration may vary form once a month to daily depending on a number of clinical factors, including the type of disease, severity of disease, and the subject's sensitivity to the therapeutic agent.

### Uses for the Antibody Variant

The antibody variants of the invention may be used as affinity purification agents. In this process, the antibodies are immobilized on a solid phase such as SEPHADEX™ resin or filter paper, using methods well known in the art. The immobilized antibody variant is contacted with a sample containing the target to be purified, and thereafter the support is washed with a suitable solvent that will remove substantially all the material in the sample except the target to be purified, which is bound to the immobilized antibody variant. Finally, the support is washed with another suitable solvent, such as glycine buffer, that will release the target from the antibody variant.

The variant antibodies may also be useful in diagnostic assays, e.g., for detecting expression of a target of interest in specific cells, tissues, or serum. For diagnostic applications, the antibody variant typically will be labeled with a detectable moiety. Numerous labels are available Techniques for quantifying a change in fluorescence are described above. The chemiluminescent substrate becomes electronically excited by a chemical reaction and may then emit light which can be measured (using a chemiluminometer, for example) or donates energy to a fluorescent acceptor. Examples of enzymatic labels include luciferases (e.g., firefly luciferase and bacterial luciferase; U.S. Pat. No. 4,737,456), luciferin, 2,3-dihydrophthalazinediones, malate dehydrogenase, urease, peroxidase such as horseradish peroxidase (HRPO), alkaline phosphatase, .beta.-galactosidase, glucoamylase, lysozyme, saccharide oxidases (e.g., glucose oxidase, galactose oxidase, and glucose-6-phosphate dehydrogenase), heterocyclic oxidases (such as uricase and xanthine oxidase), lactoperoxidase, microperoxidase, and the like. Techniques for conjugating enzymes to antibodies are described in O'Sullivan et al., Methods for the Preparation of Enzyme-Antibody Conjugates for Use in Enzyme Immunoassay, in Methods in Enzym. (Ed. J. Langone & H. Van Vunakis), Academic press, New York, 73: 147-166 (1981).

Sometimes, the label is indirectly conjugated with the antibody variant. The skilled artisan The skilled artisan will be aware of various techniques for achieving this. For example, the antibody variant can be conjugated with biotin and any of the three broad categories of labels mentioned above can be conjugated with avidin, or vice versa. Biotin binds selectively to avidin and thus, the label can be conjugated with the antibody variant in this indirect manner: Alternatively, to achieve indirect conjugation of the label with the antibody variant, the antibody variant is conjugated with a small hapten (e.g. digloxin) and one of the different types of labels mentioned above is conjugated with an anti-hapten antibody variant (e.g. anti-digloxin antibody). Thus, indirect conjugation of the label with the antibody variant can be achieved.

In another embodiment of the invention, the antibody variant need not be labeled, and the presence thereof can be detected using a labeled antibody which binds to the antibody variant.

The antibodies of the present invention may be employed in any known assay method, such as competitive binding assays, direct and indirect sandwich assays, and immunoprecipitation assays. Zola, Monoclonal Antibodies: A Manual of Techniques, pp. 147-158 (CRC Press, Inc. 1987).

Competitive binding assays rely on the ability of a labeled standard to compete with the test sample for binding with a limited amount of antibody variant. The amount of target in the test sample is inversely proportional to the amount of standard that becomes bound to the antibodies. To facilitate determining the amount of standard that becomes bound, the antibodies generally are insolubilized before or after the competition. As a result, the standard and test sample that are bound to the antibodies may conveniently be separated from the standard and test sample which remain unbound.

Sandwich assays involve the use of two antibodies, each capable of binding to a different immunogenic portion, or epitope, or the protein to be detected. In a sandwich assay, the test sample to be analyzed is bound by a first antibody which is immobilized on a solid support, and thereafter a second antibody binds to the test sample, thus forming an insoluble three-part complex. See e.g., U.S. Pat. No. 4,376,110. The second antibody may itself be labeled with a detectable moiety (direct sandwich assays) or may be measured using an anti-immunoglobulin antibody that is labeled with a detectable moiety (indirect sandwich assay). For example, one type of sandwich assay is an ELISA assay, in which case the detectable moiety is an enzyme.

For immunohistochemistry, the tumor sample may be fresh or frozen or may be embedded in paraffin and fixed with a preservative such as formalin, for example.

The antibodies may also be used for in vivo diagnostic assays. Generally, the antibody variant is labeled with a radionucleotide (such as .sup.111In, .sup.99Tc, .sup.14C, .sup.131I, .sup.3H, sup.32P or .sup.35S) so that the tumor can be localized using immunoscintiography. For example, a high affinity anti-IgE antibody of the present invention may be used to detect the amount of IgE present in, e.g., the lungs of an asthmatic patient.

The antibody of the present invention can be provided in a kit, i.e., packaged combination of reagents in predetermined amounts with instructions for performing the diagnostic assay. Where the antibody variant is labeled with an enzyme, the kit may include substrates and cofactors required by the enzyme (e.g., a substrate precursor which provides the detectable chromophore or fluorophore). In addition, other additives may be included such as stabilizers, buffers (e.g., a block buffer or lysis buffer) and the like. The relative amounts of the various reagents may be varied widely to provide for concentrations in solution of the reagents which substantially optimize the sensitivity of the assay. Particularly, the reagents may be provided as dry powders, usually lyophilized, including excipients which on dissolution will provide a reagent solution having the appropriate concentration.

### In Vivo Uses for the Antibody

It is contemplated that the antibodies of the present invention may be used to treat a mammal. In one embodiment, the antibody is administered to a nonhuman mammal for the purposes of obtaining preclinical data, for example. Exemplary nonhuman mammals to be treated include nonhuman:primates, dogs, cats, rodents and other mammals in which preclinical studies are performed. Such mammals may be established animal models for a disease to be treated with the antibody or may be used to study toxicity of the antibody of interest. In each of these embodiments, dose escalation studies may be performed on the mammal.

The antibody or polypeptide is administered by any suitable means, including parenteral, subcutaneous, intraperitoneal, intrapulmonary, and intranasal, and, if desired for local immunosuppressive treatment, intralesional administration. Parenteral infusions include intramuscular, intravenous, intraarterial, intraperitoneal, or subcutaneous administration. In addition, the antibody variant is suitably administered by pulse infusion, particularly with declining doses of the antibody variant. Preferably the dosing is given by injections, most preferably intravenous or subcutaneous injections, depending in part on whether the administration is brief or chronic.

For the prevention or treatment of disease, the appropriate dosage of the antibody or polypeptide will depend on the type of disease to be treated, the severity and course of the disease, whether the antibody variant is administered for preventive or therapeutic purposes, previous therapy, the patient's clinical history and response to the antibody variant, and the discretion of the attending physician. The very high affinity anti-human IgE antibodies of the invention may be suitably administered to the patient at one time or over a series of treatments.

Depending on the type and severity of the disease, about 0.1 mg/kg to 150 mg/kg (e.g., 0.1-20 mg/kg) of antibody is an initial candidate dosage for administration to the patient, whether, for example, by one or more separate administrations, or by continuous infusion. A typical daily dosage might range from about 1 mg/kg to 100 mg/kg or more, depending on the factors mentioned above. For repeated administrations over several days or longer, depending on the condition, the treatment is sustained until a desired suppression of disease symptoms occurs. However, other dosage regimens may be useful. The progress of this therapy is easily monitored by conventional techniques and assays. An exemplary dosing regimen for an anti-LFA-1 or anti-ICAM-1 antibody is disclosed in WO 94/04188.

The antibody variant composition will be formulated, dosed and administered in a manner consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The “therapeutically effective amount” of the antibody variant to be administered will be governed by such considerations, and is the minimum amount necessary to prevent, ameliorate, or treat a disease or disorder. The antibody variant need not be, but is optionally formulated with one or more agents currently used to prevent or treat the disorder in question. The effective amount of such other agents depends on the amount of antibody present in the formulation, the type of disorder or treatment, and other factors discussed above. These are generally used in the same dosages and with administration routes as used hereinbefore or about from 1 to 99% of the heretofore employed dosages.

The antibodies of the present invention which recognize IgE as their target may be used to treat “IgE-mediated disorders”. These include diseases such as asthma, allergic rhinitis & conjunctivitis (hay fever), eczema, urticaria, atopic dermatitis, and food allergies. The serious physiological condition of anaphylactic shock caused by, e.g., bee stings, snake bites, food or medication, is also encompassed under the scope of this invention.

### Antibody Epitope Mapping

The term “epitope” refers to a site on an antigen to which B and/or T cells respond. B-cell epitopes can be formed both from contiguous amino acids or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, and more usually, at least 5 or 8-10 amino acids in a unique spatial conformation. Antibodies that recognize the same epitope can be identified in a simple immunoassay showing the ability of one antibody to block the binding of another antibody to a target antigen.

Epitope mapping of the binding site within IgE that the high affinity anti-IgE antibodies of the present invention recognized involved Western blot analysis for binding, a peptide scan of the CH3 domain of IgE, an alanine scan of the regions that showed binding, amino acid replacement from corresponding regions of IgG1 and site directed mutagenesis.

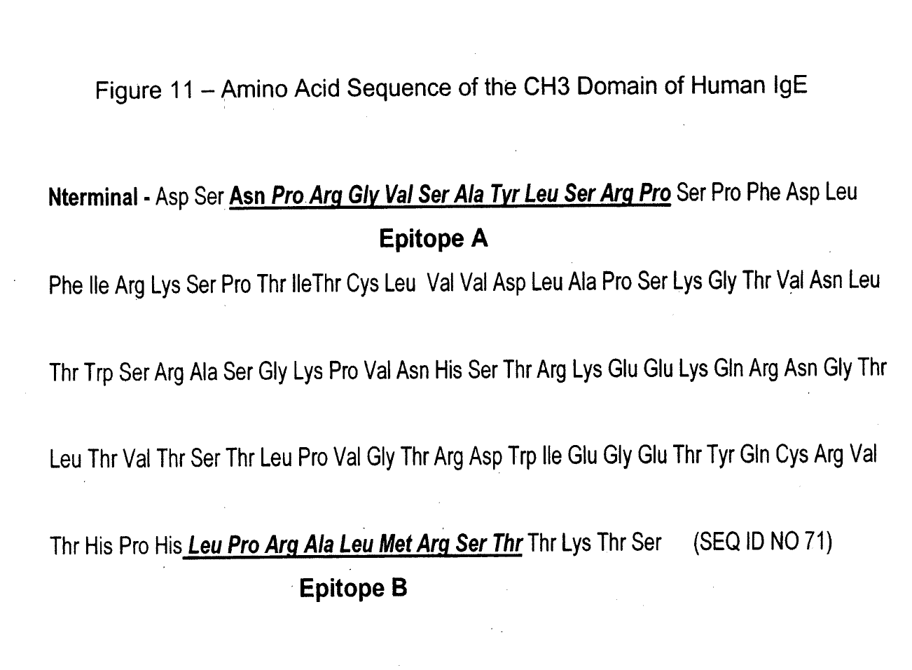

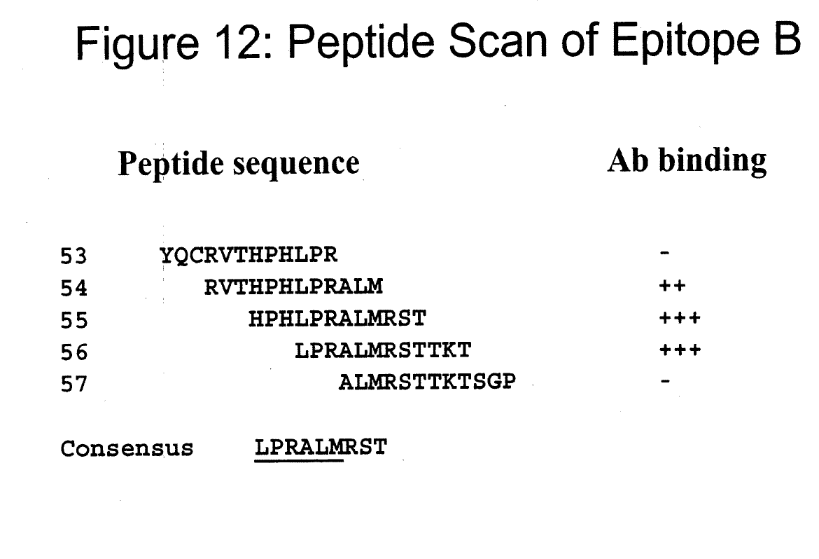

The peptide scan of the entire CH3 domain of IgE required seventy-three overlapping peptides. Each peptide was subjected to binding by labeled high affinity anti-IgE antibodies of the present invention to determine the specific epitope(s) of IgE that block binding of IgE to its high affinity receptor. The peptide scan identified two peptides as potential anti-IgE MAb contact sites on IgE, designated Epitope ‘A’ and Epitope ‘B’ (See FIG. 11). Although the Epitope ‘A’ and Epitope ‘B’ sequences are about 80 amino acids apart in the linear sequence, they are positioned close to each other in the three dimensional structure of IgE. Both are surface exposed, they overlap the FcεRI binding site of IgE, and in both peptides, there are positively charged residues of Arg and hydrophobic residues of Pro. FIG. 12 illustrates the binding region of Epitope B as determined by ELISA using peptide scan.

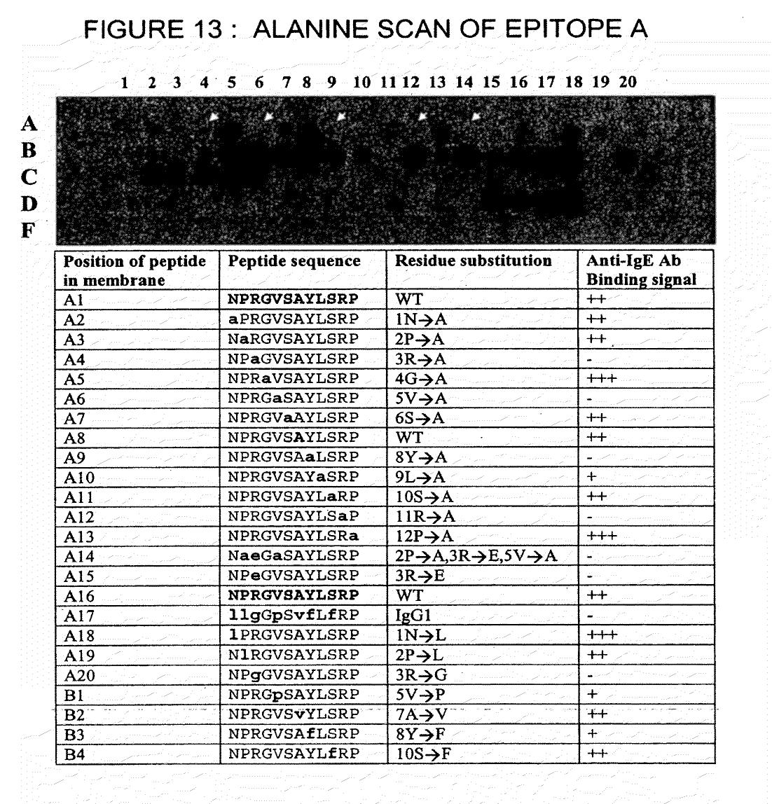

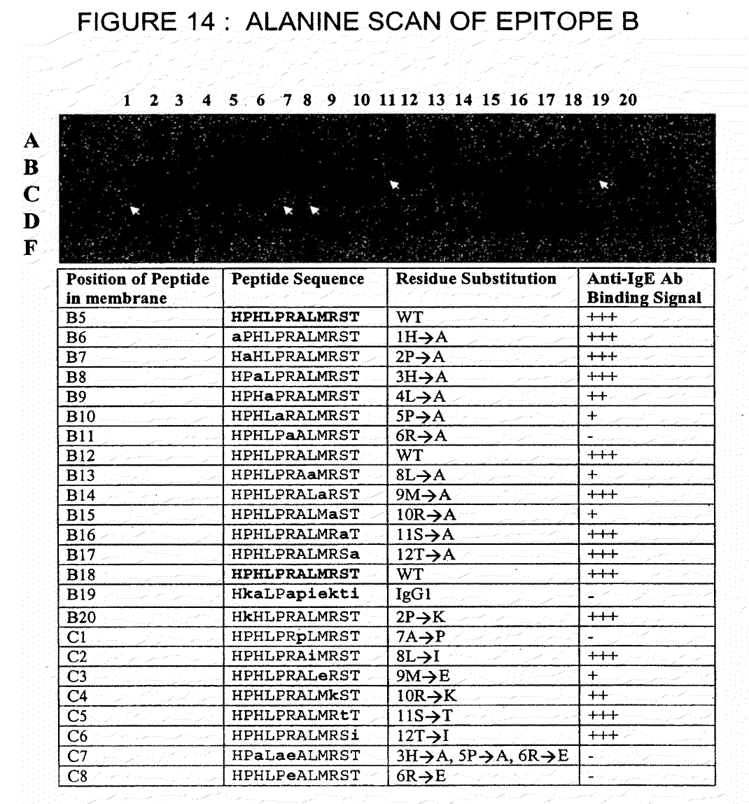

A determination of which amino acid residues are critical for binding of high affinity antibodies within these epitopes was performed by alanine scanning mutagenesis. (Cunningham et al., “High-Resolution Epitope Mapping of hGH-Receptor Interactions by Alanine-Scanning Mutagenesis” Science 244:1081-1085). Alanine was substituted for each residue of Epitope A and Epitope B and binding of high affinity monoclonal antibodies was determined. (See Example 12 below and FIGS. 13 and 14).

### Active and Passive Immunization

The invention also relates to pharmaceutical compositions, e.g., vaccines, comprising the peptide immunogen molecules of the present invention, including SEQ ID NO 72 and/or SEQ ID NO 74, and diluents, excipients, adjuvants, or carriers. It further concerns a process for the preparation of an immunogen of the invention, comprising covalently coupling at least one peptide of the invention with a moiety capable of eliciting an immune response against that peptide.

It also relates to immunogenic peptides as defined above, for use as a pharmaceutical, e.g. in the treatment of IgE-mediated diseases or conditions, such as allergy and atopic dermatitis.

It further relates to a method of immunizing a mammal against IgE-mediated diseases or conditions, such as allergies and atopic dermatitis, comprising the administration of a therapeutically effective amount of the immunogenic peptides as defined above to a patient in need of such treatment.

The immunogenic peptides of the present invention, while being substantially incapable of mediating non-cytolytic histamine release, are capable of eliciting antibodies with strong serological cross-reactivity with the target amino acid sequences of Epitope A and/or Epitope B.

The initial dose of peptide (e.g. from about 0.2 mg to about 5 mg) may be administered, for example, intramuscularly, followed by repeat (booster) doses of the same at 14 to 28 days later. Doses will of course depend to some extent on the age, weight and general health of the patient. Immunization may be “active” or “passive”. In “active” immunization the subject receives an immunogenic peptide of the present invention and an anti-IgE response is actively induced by the subject's immune system.

“Active” immunization, this is preferred for human use, but other mammalian species may be treated similarly, as e.g. in the dog. The term “immunogenic carrier” herein includes those materials which have the property of independently eliciting an immunogenic response in a host animal and which can be covalently coupled to polypeptide either directly via formation of peptide or ester bonds between free carboxyl, amino or hydroxyl groups in the polypeptide and corresponding groups on the immunogenic carrier material, or alternatively by bonding through a conventional bifunctional linking group, or as a fusion protein.

Examples of such immunogenic carriers include: albumins, such as BSA; globulins; thyroglobulins; hemoglobins; hemocyanins (particularly Keyhole Limpet Hemocyanin [KLH]); proteins extracted from ascaris, e.g. ascaris extracts such as those described in J. Immunol. 111 [1973] 260-268, J. Immunol. 122 [1979] 302-308, J. Immun. 98 [1967] 893-900, a Am. J. Physiol. 199 [1960] 575-578 or purified products thereof; polylysine; polyglutamic acid; lysine-glutamic acid copolymers; copolymers containing lysine or ornithine; etc. Vaccines have been produced using diphteria toxoid or tetanus toxoid as immunogenic carrier material (Lepow M. L. et al., J. of Infectious Diseases 150 [1984] 402-406; Coen Beuvery, E. et al., Infection and Immunity 40 [1983] 39-45) and these toxoid materials can also be used in the present invention. The purified protein derivative of tuberculin (PPD) is particularly preferred for utilization in the “active” immunization scheme since (I) it does not induce a T-cell response itself (i.e. it is in effect a “T-cell hapten”), and yet behaves as a fully processed antigen and is recognized by T-cells as such; (2) it is known to be one of the most powerful hapten “carriers” in the linked recognition mode; and (3) it can be used in humans without further testing.

The present invention also relates to polynucleotides encoding the peptides of the present invention, vectors comprising said polynucleotides, and cells harboring said vectors. In addition, active immunization may be achieved by administering the polynucleotides encoding the peptides of the present invention. Vectors suitable for such therapy are known in the art and include, e.g., adenovirus vectors.

“Passive” immunization is achieved by administering anti-IgE antibodies of the present invention, to a patient suffering from IgE-mediated disease or condition.

These antibodies can be prepared by administering an immunogenic peptide of the present invention to a non-human mammal and collecting the resultant antiserum. Improved titres can be obtained by repeated injections over a period of time. There is no particular limitation to the species of mammals which may be used for eliciting antibodies; it is generally preferred to use rabbits or guinea pigs, but horses, cats, dogs, goats, pigs, rats, cows, sheep, etc., can also be used. Antibody is recovered by collecting blood from the immunized animal after the passage of 1 to 2 weeks subsequently to the last administration, centrifuging the blood and isolating serum from the blood. Monoclonal antibodies may e.g. be human or murine.

When immunizing a subject, an antibody of the present invention can be introduced into the mammal by, e.g., intramuscular injection. However, any form of antibody administration may be used. Any conventional liquid or solid vehicle may be employed which is acceptable to the subject and does not have adverse side effects. Phosphate-buffered saline (PBS), at a physiological pH, e.g. about pH 6.8 to 7.2, preferably about pH 7.0, may be used as a vehicle, alone or with a suitable adjuvant, such as an aluminium hydroxide-based adjuvant.

The following examples are offered by way of illustration and not by way of limitation.

### EXAMPLES

### Example 1

Humanization of Anti-IgE Murine MAb TES-C21

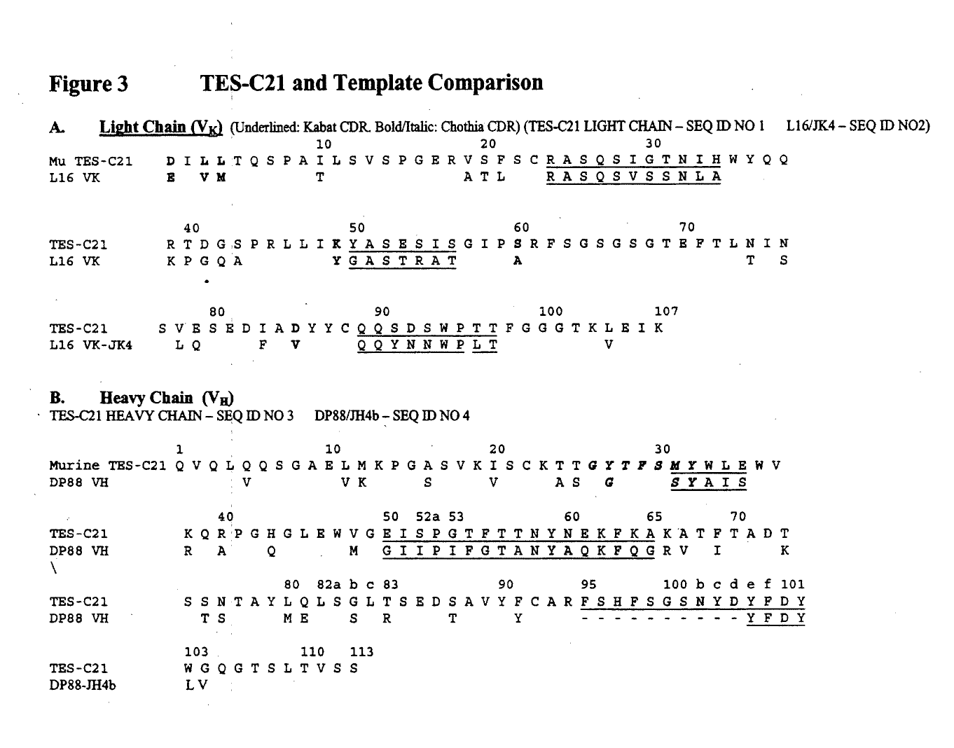

The sequences of the heavy chain variable region (V_{H}) and the light chain variable region (V_{L}) of murine mAb TES-C21 were compared with human antibody germline sequences available in the public databases. Several criteria were used when deciding on a template as described in step 1 above, including overall length, similar CDR position within the framework, overall homology, size of the CDR, etc. All of these criteria taken together provided a result for choosing the optimal human template as shown in the sequence alignment between TES-C21 MAb heavy and light chain sequences and the respective human template sequences depicted in FIGS. 3A and 3B.

In this case, more than one human framework template was used to design this antibody. The human template chosen for the V_{H} chain was a combination of DP88 (aa residues 1-95) and JH4b (aa residues 103-113) (See FIG. 3B). The human template chosen for the V_{L} chain was a combination of L16 (VK subgroup III, aa residues 1-87) combined with JK4 (aa residues 98-107) (See FIG. 3A). The framework homology between the murine sequence and the human template was about 70% for V_{H} and about 74% for V_{L}.

Once the template was chosen, a Fab library was constructed by DNA synthesis and overlapping PCR as described above and depicted in FIG. 2. The library was composed of synthesized TES-C21 CDRs synthesized with the respective chosen human templates, DP88/JH4b and L16/JK4. The complexity of the library was 4096 (=2¹²). The overlapping nucleotides encoding partial V_{H} and V_{L} sequences were synthesized in the range of about 63 to about 76 nucleotides with 18 to 21 nucleotide overlaps.

PCR amplification of V_{L} and V_{H} gene was performed using a biotinylated forward primer containing the specific sequence to the framework region FR1 and an overhanging sequence annealed to the end of leader sequence (GeneIII) and a reverse primer from the conserved constant region (Cκ or CH1) under standard PCR conditions. The PCR product was purified by agarose gel electrophoresis, or by commercial PCR purification kit to remove unincorporated biotinylated primers and non-specific PCR.

5′-Phosphorylation of PCR product was performed using 2 μg PCR product, 1 μL of T4 polynucleotide kinase (10 units/μL), 2 μL of 10×PNK buffer, 1 μL of 10 mM ATP in a total volume of 20 μL adjusted by ddH₂O. After incubating at 37° C. for 45 minutes, and heat denaturation at 65° C. for 10 min, the reaction volume was adjusted to 200 μL by adding ddH₂O for the next step.

The 100 μL of streptavidin-coated magnetic beads were washed twice with 200 μL 2×B&W buffer and resuspended in 200 μL 2×B&W buffer. The phosphorylated PCR product was mixed with beads, and incubated at room temperature (RT) for 16 min with mild shaking.

The beads were sedimented and washed twice with 200 μL 2×B&W buffer. The non-biotinylated ssDNA (minus strand) was eluted with 300 μL freshly prepared 0.15M NaOH at RT for 10 min with mild shaking. A second NaOH elution can increase the yield slightly (optional). The eluant was centrifuged to remove any trace beads.

The ssDNA was precipitated from the supernatant by adding 1 μL glycogen (10 mg/mL), 1/10 volume of 3M NaOAc (pH 5.2), and 2.5 volumes of EtOH. The precipitated ssDNA was then washed with 70% EtOH followed by lyophilizing for 3 min and dissolving in 20 μL ddH₂O. The ssDNA was quantitated by spotting on an ethidium bromide (EtBr) agarose plate with DNA standards, or by measuring OD₂₆₀.

### Example 2

### Cloning of V_{H} and V_{L} into Phage-Expression Vector

V_{H} and V_{L} were cloned into a phage-expression vector by hybridization mutagenesis. Uridinylated templates were prepared by infecting CJ236 E. coli strain (dut⁻ ung⁻) with M13-based phage (phage-expression vector TN003).

The following components [200 ng of uridinylated phage vector (8.49 kb); 92 ng phosphorylated single-stranded H chain (489 bases); 100 ng phosphorylated single-stranded L chain (525 bases); 1 μL 10× annealing buffer; adjust volume with ddH₂O to 10 μl] were annealed (at about 8-fold molar ratio of insert to vector) by PCR holding the temperature at 85° C. for 5 min (denaturation) and then ramping to 55° C. over 1 hour. The samples were chilled on ice.

To the annealed product the following components were added: 1.4 μL 10× synthesis buffer; 0.5 μL T4 DNA ligase (1 unit/μL); 1 μL T4 DNA polymerase (1 unit/μL) followed by incubating on ice for 5 min, and 37° C. for 1.5 hours. The product was then ethanol precipitated, and dissolved in 10 μL of ddH₂O or TE.

DNA was digested with 1 μL XbaI (10 unit/μL) for 2 h, and heat inactivated at 65° C. for 20 min. Digested DNA was transfected into 50 μL of electro-competent DH10B cells by electroporation. The resulting phage were titered by growing on XL-1 Blue bacterial lawn at 37° C. overnight. Clones were sequenced to confirm composition.

### Example 3

### Deep Well Culture for Library Screening

A. Plating Phage Library

The phage library was diluted in LB media to achieve the desired number of plaques per plate. High titer phage was mixed with 200 μL XL-1 B cell culture. 3 mL LB top agar was mixed, poured onto an LB plate, and allowed to sit at room temperature for 10 minutes. The plate was incubated overnight at 37° C.

B. Phage Elution

100 μL of phage elution buffer (10 mM Tris-Cl, pH 7.5, 10 mM EDTA, 100 mM NaCl) was added to each well of a sterile U-bottom 96 well plate. A single phage plaque from the overnight library plate was transferred with a filtered pipette tip to a well. The phage elution plate was incubated at 37° C. for 1 hour. The plate may be stored at 4° C. following incubation.

C. Culture for Deep Well Plates

XL1B cells from 50 mL culture were added to 2×YT media at a 1:100 dilution. The cells were grown at 37° C. in a shaker until the A₆₀₀ was between 0.9 to 1.2.

C. Infection with Phage in Deep Well Plates

When the cells reached the appropriate OD, 1M IPTG (1:2000) was added to the XL1B culture. The final concentration of IPTG was 0.5 mM. 750 μL of cell culture was transferred to each well of a 96 well-deep well plate (Fisher Scientific). Each well was inoculated with 25 μL of eluted phage. The deep well plate was placed in the shaker (250 rpm) and incubated overnight at 37° C.

D. Preparing Supernatant for ELISA Screening

Following incubation, the deep well plates were centrifuged at 3,250 rpm for 20 minutes using the Beckman JA-5.3 plate rotor. 50 μL of supernatant was withdrawn from each well for ELISA.

E. Innoculation of 15 mL Liquid Cultures of XL-1 Cells

XL-1s were grown at 37° C. in the shaker (250 rpm) in 2×YT containing 10 μg/mL of tetracycline until A₆₀₀=0.9 to 1.2. IPTG was added at a final concentration of 0.5 mM and 15 mL of the culture was transferred to a 50 mL conical tube for each clone to be characterized. The cells were inoculated with 10 μL of phage from the high titer stock (titer=˜10¹¹ pfu/mL) and incubated for 1 hour at 37° C. The cells were grown overnight at room temperature with shaking.

F. Isolation of Soluble Fab from Periplasm

The cells were pelleted in an IEC centrifuge at 4,500 rpm for 20 minutes. Culture medium was removed the pellet was resuspend in 650 μL of resuspension buffer (50 mM Tris, pH 8.0 containing 1 mM EDTA and 500 mM sucrose), vortexed, and placed on ice for 1 hour with gentle shaking. Cellular debris was removed by centrifugation at 9,000 rpm for 10 minutes at 4° C. The supernatant containing the soluble Fabs was collected and stored at 4° C.

### Example 4

### Framework Modification

There were twelve murine/human wobble residues within the framework at the potential key positions described above. Position 73 in V_{H} was kept as the murine residue threonine in the humanization library because this position was determined to affect binding. It was noted, however, that threonine at VH 73 is a common human residue in the human germline V_{H} subgroup 1 and 2.

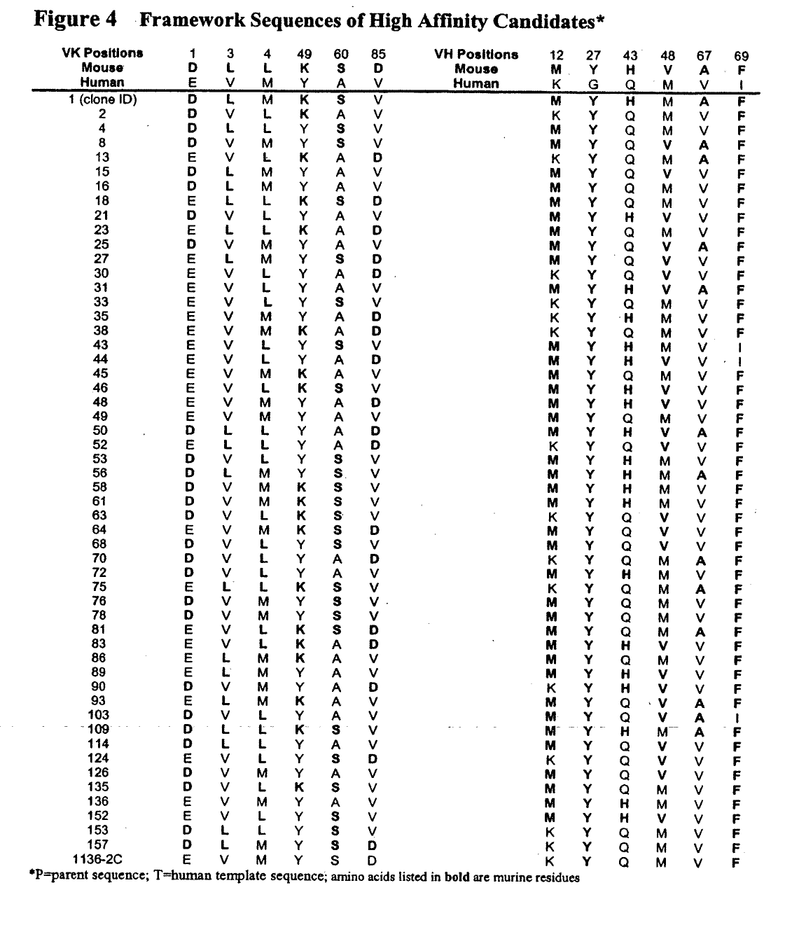

The framework residues that differed between the TES-C21 sequence and the human template were randomly substituted as described above and then assessed for their potential affect on target binding, and antibody folding. Potential framework residues that may have affected the binding were identified. In this case, they were residues 12, 27, 43, 48, 67, 69 in V_{H}, and 1, 3, 4, 49, 60, 85 in V_{L} (Kabat number system). (See FIG. 4) It was later demonstrated that only positions 27 and 69 significantly affected binding in the V_{H} region (clone number 1136-2C).

The primary screen used was a single point ELISA (SPE) using culture media (See description below). The primary screen selected clones that that bind to the antibody's target molecule. Clones that gave equal or better signal than the parent molecule were selected for the next round of screening.

In the second round of screening, individual phage were grown in a 15 ml bacterial culture and periplasmic preparations were used for SPE and ELISA titration assays. The clones that retained higher binding in this assay were further characterized. Once all the selected primary clones were processed, the top 10-15% clones were sequenced and the clones arranged according to sequence. Representatives from each sequence group were compared against each other and the best clones selected. Sequences from these chosen clones were combined and the effects of various combinations were evaluated.

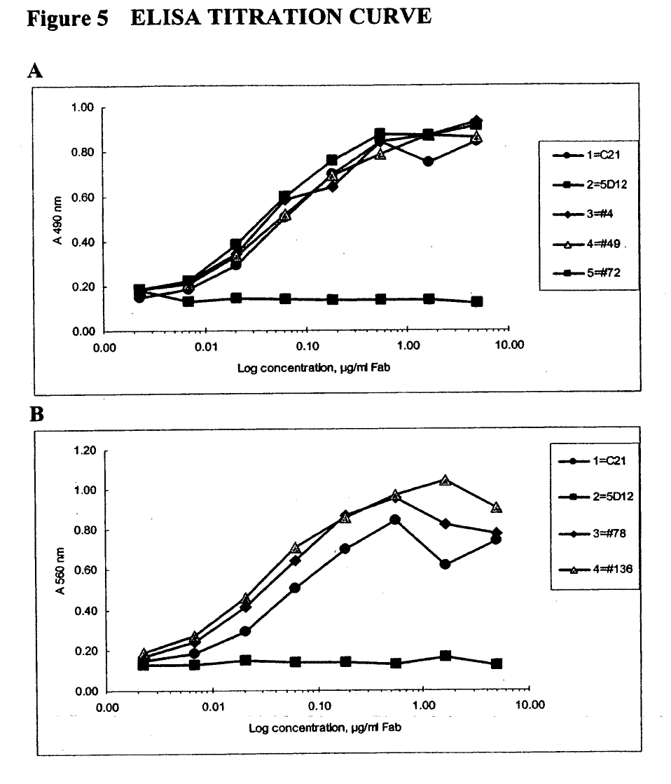

The constructed library was subjected to an ELISA screen for improved binding to the recombinant human IgE, SE44. Clones with binding affinity greater than murine TES-C21 Fab were isolated and sequenced. Clone ID #4, 49, 72, 76, and 136 were further characterized. ELISA titration curves for clone 4, 49, 72, 78, and 136 are shown in FIGS. 5A and 5B indicating that their affinity is similar to the parent, TES-C21. These clones compete with murine TES-C21 for binding to human IgE indicating that the binding epitope was not changed during the humanization process. The humanized Fabs did not bind to FcεRI-bound IgE suggesting that it is less likely that the humanized antibodies will crosslink the receptor to cause histamine release when they were constructed into divalent IgG.

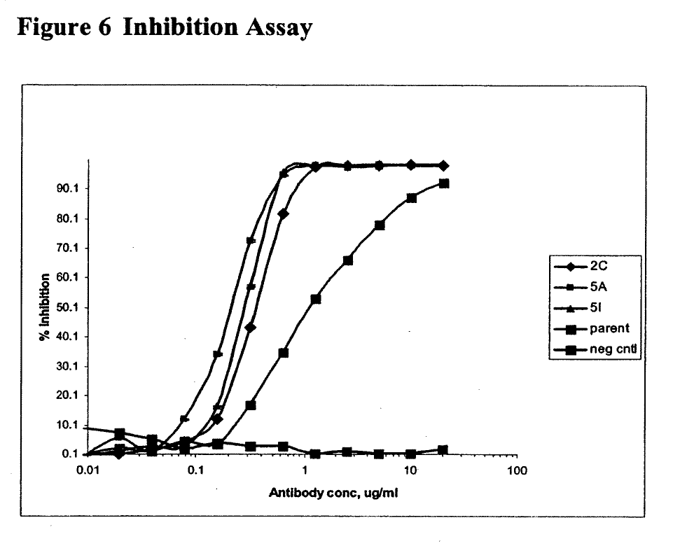

Humanized clone 136 retained 5 murine heavy chain framework residues (=94.3% human V_{H} framework homology), with a 100% human light chain framework selected for by affinity maturation. The inhibition of IgE binding to FcεRI by the humanized Fab was demonstrated (FIG. 6).

### Example 5

### Single Point ELISA Protocol for Screening Anti IgE

Plates were coated with 2 ug/mL sheep anti-human Fd in carbonate coating buffer overnight at 4° C. The coating solution was removed and the plates were blocked with 200 uL/well 3% BSA/PBS for 1 hour at 37° C. After washing the plates 4× with PBS/0.1% TWEEN® (PBST), 50 uL/well Fab sample (i.e., supernatant containing high titer phage and secreted Fab or periplasmic prep from DMB block, or 15 mL prep) was added. Plates were incubated for 1 hour at room temperature followed by washing 4× with PBST. 50 uL/well of biotinylated SE44 at 0.015 ug/mL diluted in 0.5% BSA/PBS and 0.05% TWEEN®was then added. Plates were then incubated for 2 hours at room temperature and washed 4×PBST. 50 uL/well StreptAvidin HRP 1:2000 dilution in 0:5% BSA/PBS and 0.05% TWEEN® was added and the plates incubated 1 hour at room temperature. Plates were washed 6× with PBST. 50 uL/well TMB substrate (sigma) was added to develop and then stopped by adding 50 uL/well 0.2M H₂SO₄.

### Example 6

### ELISA Titration

### Anti IgE

Plates were coated with 0.25 ug/mL (for purified Fab 0.1 ug/ml) SE44 in carbonate coating buffer overnight at 4° C. Coating solution was removed and the plates were blocked with 200 uL/well 3% BSA/PBS for 1 hour at 37° C.

The plates were washed 4× with PBS/0.1% TWEEN® (PBST). 50 uL/well Fab (from 15 mL periplasmic prep) was added starting with a dilution of 1:2 and diluting 3 fold serially in 0.5% BSA/PBS and 0.05% TWEEN®20. Plates were incubated for 2 hours at room temperature.

The plates were washed 4× with PBST and 50 uL/well 1:1000 (0.8 ug/ml) dilution of biotin-sheep anti human Fd in 0.5% BSA/PBS and 0.05% TWEEN®20 was added. The plates were incubated again for 2 hours at room temperature.

Following a wash 4× with PBST, 50 uL/well Neutra-avidin-AP1: 2000(0.9 ug/ml) in 0.5% BSA/PBS and 0.05% TWEEN® 20 was added and the plates were incubated 1 hour at room temperature.

The plates were washed 4× with PBST. And developed by adding 50 uL/well pNPP substrate. Development was stopped by adding 50 uL/well 3M NaOH. The absorbance of each well was read at 405 nm or 410 nm.

### Example 7

### Protocol for Affinity Purification of M13 Phage Expressed Soluble Fab

A. Day 1

Two 500 mL cultures (2×YT) containing 10 mg/mL tetracycline were innoculated with 5 mL overnight stock XL1 B and grown at 37° C. to A600=0.9 to 1.2. IPTG was added to a concentration of 0.5 mM. The cell culture was then infected with 200 μL phage per culture and incubated for 1 hour at 37° C. with shaking. Following infection, the cells were grown at 25° C. overnight with shaking.

B. Day 2

Cells were pelleted at 3500×g for 30 minutes at 4° C. in 250 mL centrifuge tubes. Culture medium was aspirated and the pellets were resuspended in a total of 12-15 mL lysis buffer (Buffer A+protease inhibitor cocktail).

Buffer A: (1 Liter)
50 mM NaH₂PO₄ 6.9 g NaH₂PO₄H₂O (or 6 g NaH₂PO₄)
300 mM NaCl 17.54 g NaCl
10 mM imidazole 0.68 g imidazole (MW 68.08)
adjust pH to 8.0 using NaOH

Lysis Buffer:
Mix 25 mL of Buffer A with one tablet of Complete Protease Inhibitor Cocktail (Roche, Basel, Switzerland).

Resuspended cells were transferred into a 50 mL conical tube and lysed with 100 μL 100 mg/mL lysozyme by inverting the tube several times until the mixture moves together as a blob (due to the lysis). Cells were sonicated on ice followed by the addition of 10 μL DNase I (about 1000 units) and gently rocked at 4° C. for 30 minutes. Debri was pelleted by centrifugation at 12000×g for 30 minutes at 4° C., using 50 mL centrifuge tubes. Supernatants were transferred to a new conical tube and stored at 4° C.

Ni-NT agarose (Qiagen, Valencis, Calif.) was used to purify the soluble Fabs according to the manufacturer's protocol. The lysate was mixed with Ni-NTA and loaded into a column. The flow through was collected for SOS-PAGE analysis. The column was washed with 20 mL buffer (50 mM NaH₂PO₄, 300 mM NaCl, 15 mM imidazole, adjust pH to 8.0 with NaOH) followed by a 20 mL wash with 50 mM NaH₂PO₄, 300 mM NaCl, 20 mM imidazole. Fabs were eluted with 6×500 μL elution buffer (50 mM NaH₂PO₄, 300 mM NaCl, 450 mM imidazole, adjust pH to 8.0 with NaOH) and analyzed by SDS PAGE. Column fractions were stored at 4° C. Column fractions were analyzed by SDS-PAGE and the fraction with the greatest amount of Fab was selected and dialyzed in PBS at 4° C.

### Example 8

### Soluble Receptor Assay

A 96 well assay plate suitable for ELISA was coated with 0.05 mL 0.5 μg/mL FcεRI alpha-chain receptor coating buffer (50 mM carbonate/bicarbonate, pH 9.6) for 12 hours at 4-8° C. The wells were aspirated and 250 μL blocking buffer (PBS, 1% BSA, pH 7.2) was added and incubated for 1 hour at 37° C. In a separate assay plate the samples and reference TES-C21 MAbs were titered from 200 to 0.001 μg/mL by 1:4 dilutions with assay buffer (0.5% BSA and 0.05% Tween 20, PBS, pH 7.2) and an equal volume of 100 ng/mL biotinylated IgE was added and the plate incubated for 2-3 hours at 25° C. The FcεRI-coated wells were washed three times with PBS and 0.05% TWEEN 20 and 50 μL from the sample wells were transferred and incubated with agitation for 30 minutes at 25° C. Fifty μL/well of 1 mg/mL Streptavidin-HRP, diluted 1:2000 in assay buffer, was incubated for 30 minutes with agitation and then the plate was washed as before. Fifty μL/well of TMB substrate was added and color was developed. The reaction was stopped by adding an equal volume of 0.2 M H₂SO₄ and the absorbance measured at 450 nm.

### Example 9

### Binding of Antibodies to IgE-Loaded FcεRI

Antibody binding to human IgE associated with the alpha-subunit of FcεRI was determined by preincubating with 10 μg/mL human IgE for 30 min at 4° C. Plates were washed three times followed by a one hour incubation with varying concentrations of either murine anti-human IgE MAb E-10-10 or the humanized Fab variant. Binding of Fabs was detected with a biotin labeled anti human Fd antibody followed by SA-HRP. Murine Mab E-10-10 was detected by Goat anti-murine Ig Fc HRP-conjugated Ab.

### Example 10

### Clone Characterization

Each candidate was assayed for binding affinity and positive clones were sequenced. Antibody variants having beneficial mutations in CDR regions that increase binding affinity were further characterized. Assays included Biacore analysis; inhibition of IgE binding to its receptor; and cross linking of receptor bound IgE.

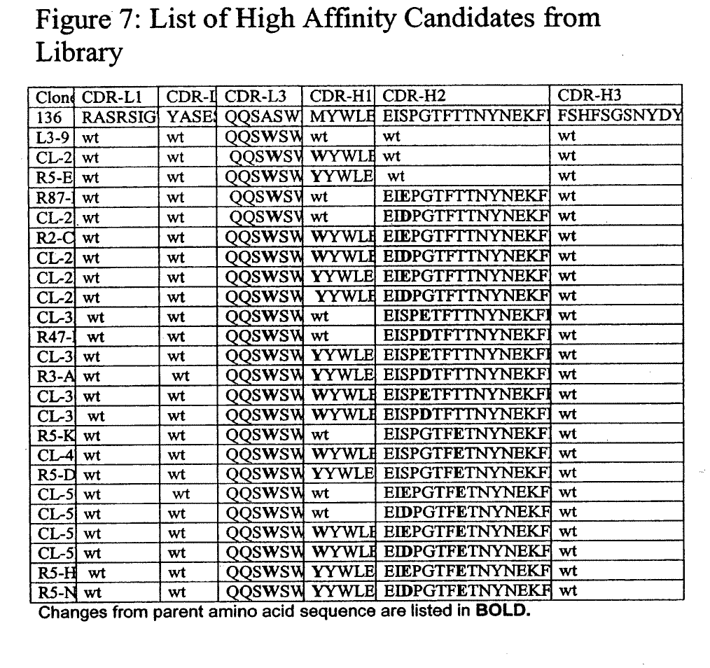

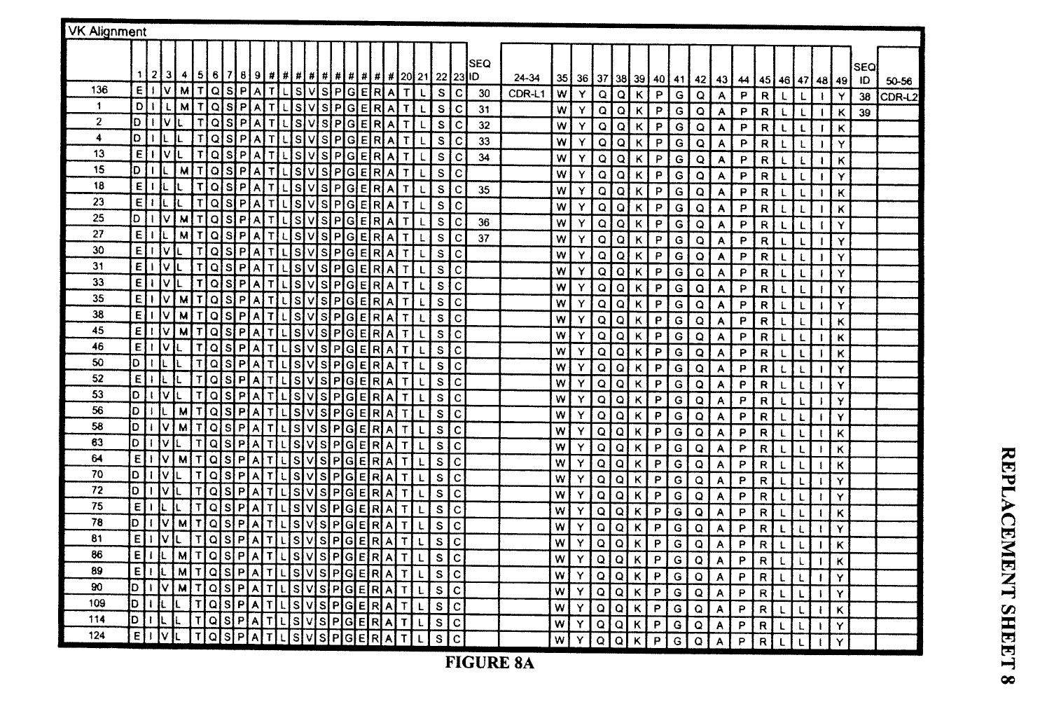

A library of variants was created. The amino acid sequences for the various CDRs which demonstrated improved affinity are depicted in Table 1. FIG. 7 presents high affinity candidates having combinations of substitutions.

TABLE 1
CDRL1:

| P | RASQSIGTNIH | SEQ ID NO 5 |
|---|---|---|
| #1 | RASRSIGTNIH | SEQ ID NO 6 |
| #2 | RASQRIGTNIH | SEQ ID NO 7 |

CDRL2:

| | | |
|---|---|---|
| P | YASESIS | SEQ ID NO 8 |
| #1 | YAYESIS | SEQ ID NO 9 |
| #2 | YASESIY | SEQ ID NO 10 |
| #3 | YASESDS | SEQ ID NO 11 |
| #4 | YASESES | SEQ ID NO 12 |

CDRL3:

| | | |
|---|---|---|
| P | QQSDSWPTT | SEQ ID NO 13 |
| #1 | AASWSWPTT | SEQ ID NO 14 |

CDRH1:

| | | |
|---|---|---|
| P | MYWLE | SEQ ID NO 15 |
| #1 | WYWLE | SEQ ID NO 16 |
| #2 | YYWLE | SEQ ID NO 17 |

CDRH2:

| | | |
|---|---|---|
| P | EISPGTFTTNYNEKFKA | SEQ ID NO 18 |
| #1 | EIEPGTFTTNYNEKFKA | SEQ ID NO 19 |
| #2 | EIDPGTFTTNYNEKFKA | SEQ ID NO 20 |
| #3 | EISPDTFTTNYNEKFKA | SEQ ID NO 21 |
| #4 | EISPETFTTNYNEKFKA | SEQ ID NO 22 |
| #5 | EISPGTFETNYNEKFKA | SEQ ID NO 23 |
| #6 | EIEPGTFETNYNEKFKA | SEQ ID NO 24 |
| #7 | EIDPGTFETNYNEKFKA | SEQ ID NO 25 |

CDRH3:

| | | |
|---|---|---|
| P | FSHFSGSNYDYFDY | SEQ ID NO 26 |
| #1 | FSHFSGMNYDYFDY | SEQ ID NO 27 |
| #2 | FSHFSGQNYDYFDY | SEQ ID NO 28 |
| #3 | FSHFTGSNYDYFDY | SEQ ID NO 29 |

P = Parent

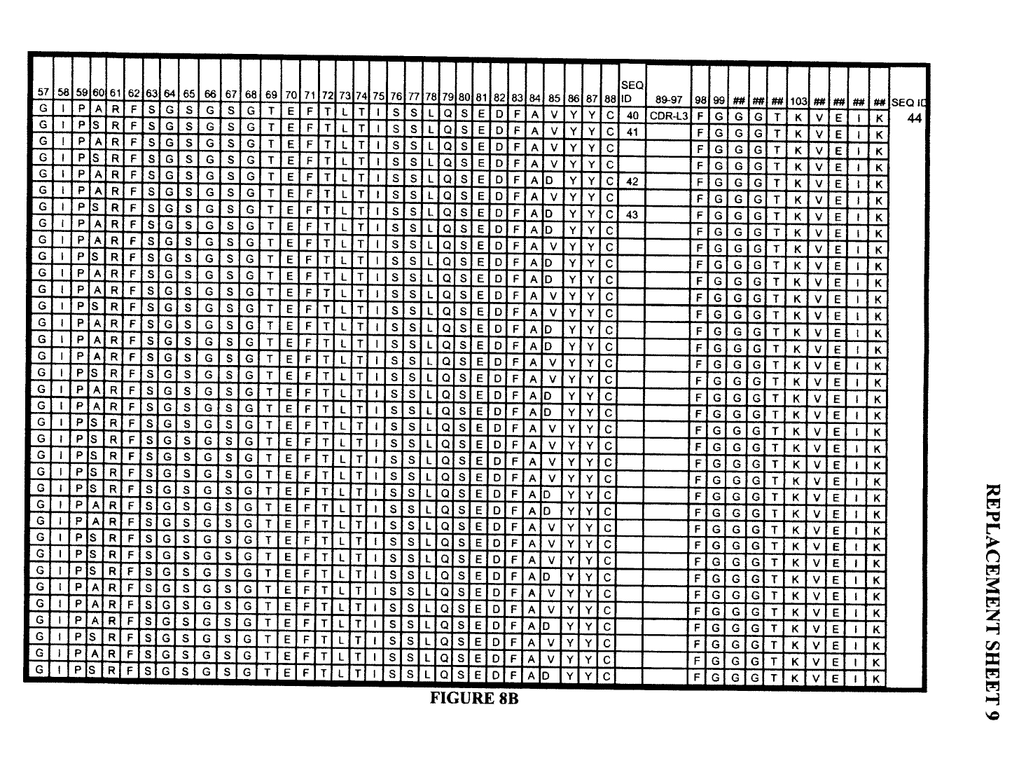

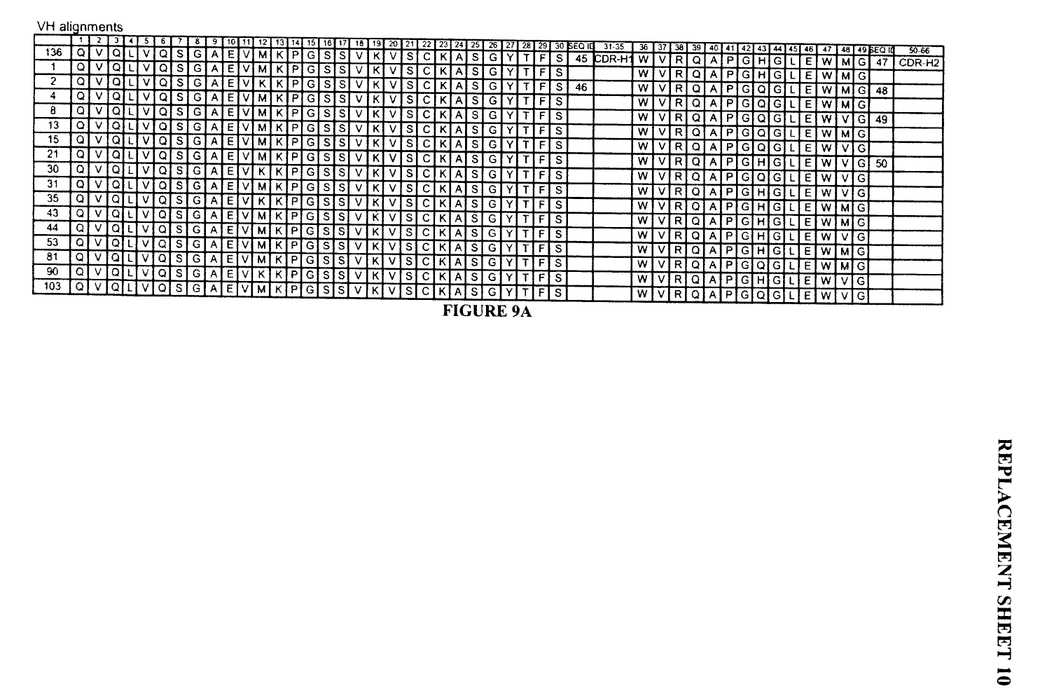

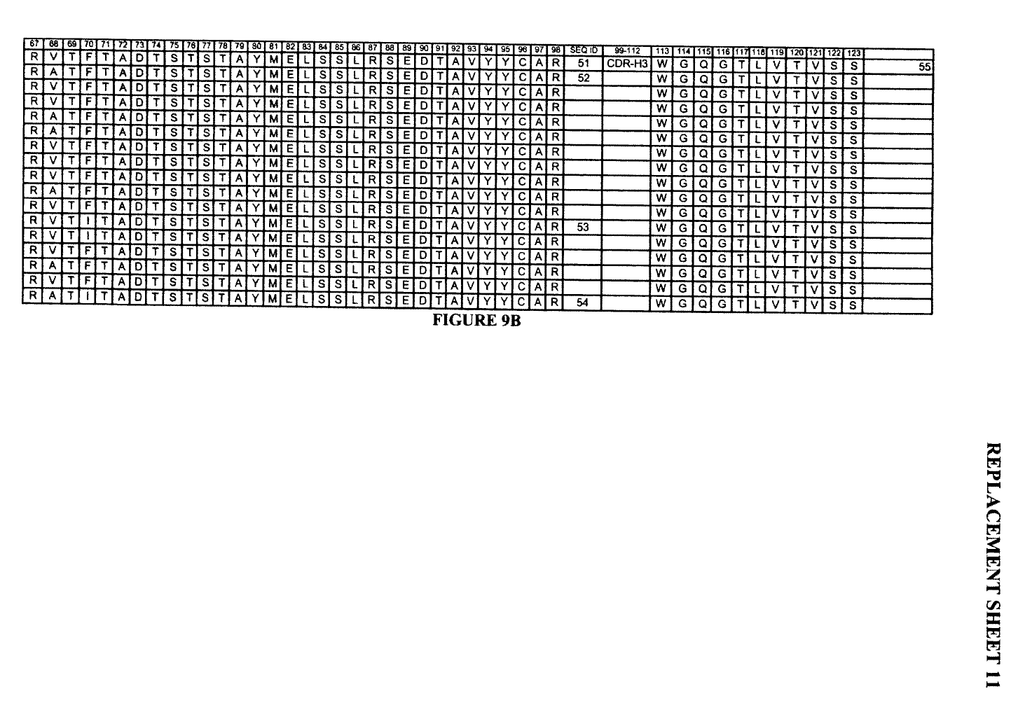

Nineteen heavy chain variants are presented in FIG. 9 and 35 light chain variants are presented in FIG. 8. Three candidates were further characterized for binding affinity and these are presented in Table 2.

TABLE 2
Binding Affinity

| | | |
|---|---|---|
| | | Fold Increase in |
| MAb | K_{D} | Binding Affinity |

| | | | |
|---|---|---|---|
| TES-C21 | 614 ± 200 | pM | |
| MAb 1(CL-5A) | 0.158 | pM | 3886 |
| MAb 2 (CL-2C) | 1.47 ± 0.5 | pM | 417 |
| MAb 3 (CL-5I) | 3.2 ± 2.2 | pM | 191 |

### Example 11

### Expression and Purification of Anti-IgE Antibodies and HRP-Conjugation

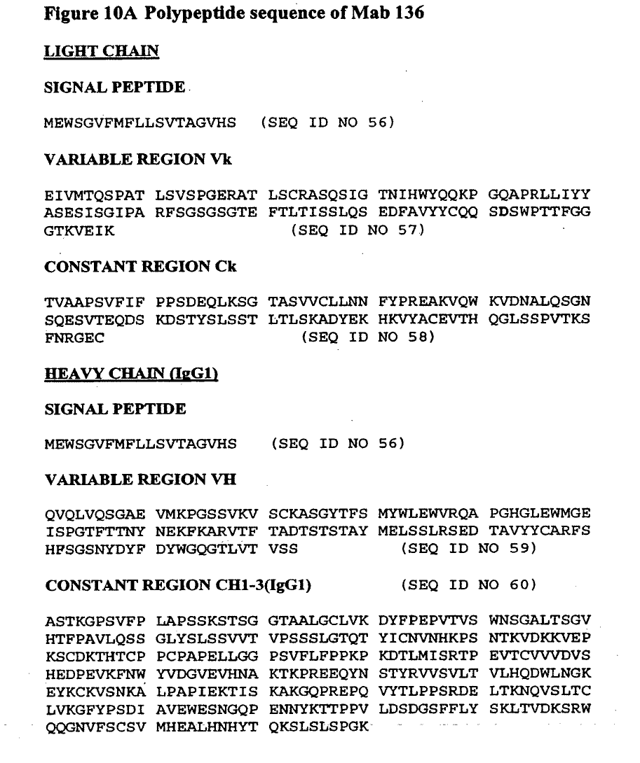

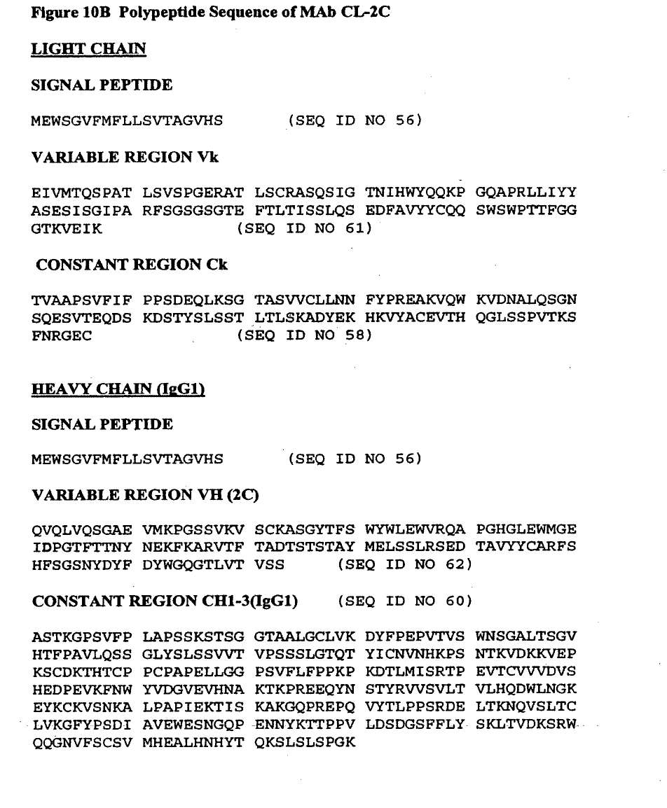

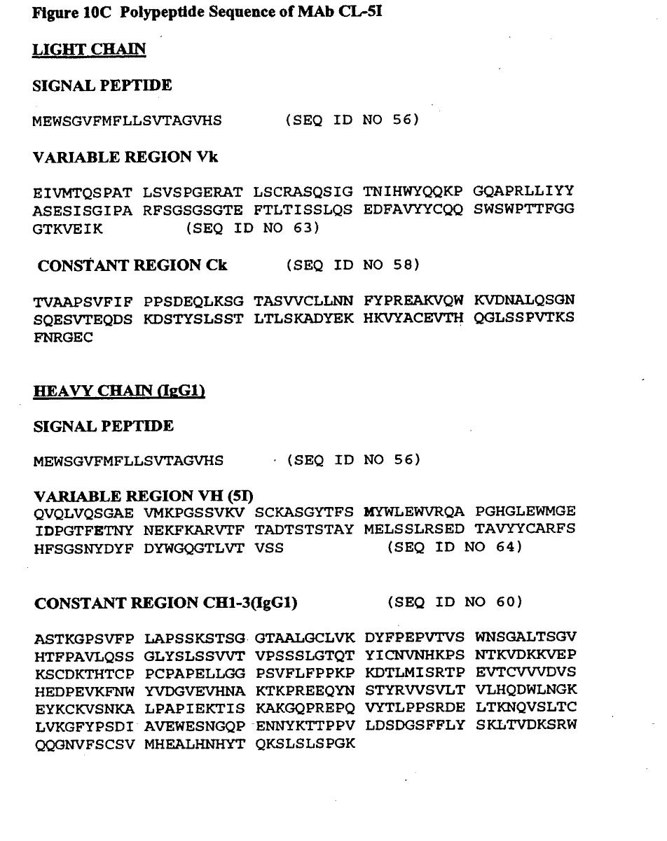

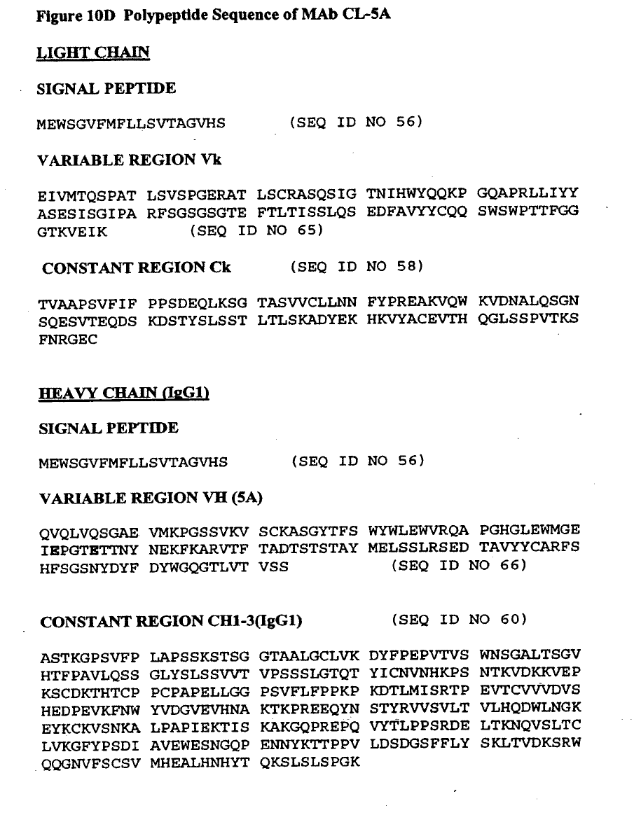

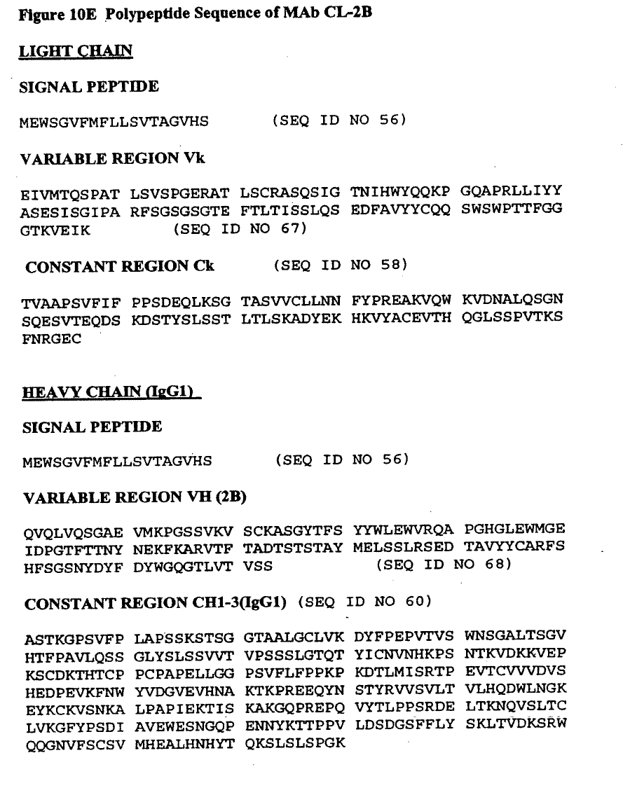

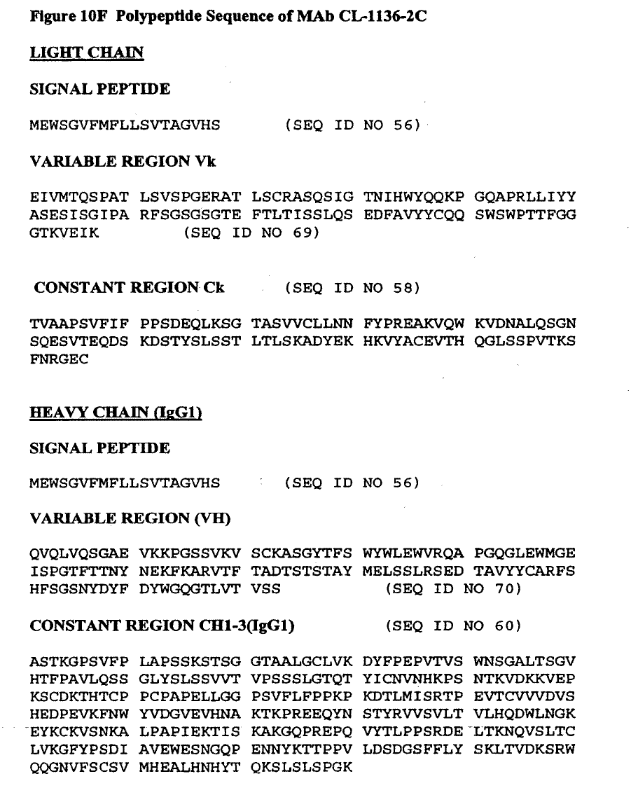

High affinity MAbs candidates were generated. For the generation of intact anti-IgE MAbs, the heavy and light chains variable regions were PCR amplified from phage vectors templates and subcloned separately into H- and L-chain expression vectors under the expression of a CMV promoter. Six full antibody clones were constructed and are represented in FIG. 10 A-F. Appropriate heavy and light chain plasmids were co-transfected into the mouse myeloma cell line NSO using electroporation by techniques well known in the art. See, e.g., Liou et al. J Immunol. 143(12):3967-75 (1989). Antibodies were purified from the single stable cell line supernatants using protein A-sepharose (Pharmacia). The concentration of the antibody was determined using spectrophotometer at 280 nm and FCA assay (IDEXX).

Purified antibodies were conjugated by horseradish peroxidase (HRP) using peroxidase conjugation kit (Zymed Labs, San Francisco, Calif.) according to the manufacturer's protocol. The titer of each conjugated anti-IgE MAb was determined using ELISA with plates coated with a monoclonal human IgE (SE44).

The following cultures have been deposited with the American Type Culture Collection, 10801 University Boulevard, Manassas Va. 20110-2209 USA (ATCC):

| Hybridoma | ATCC NO. | Deposit Date |
|---|---|---|
| Anti-IgE CL-2C | PTA-5678 | Dec. 3, 2003 |
| Anti-IgE CL-5A | PTA-5679 | Dec. 3, 2003 |
| Anti-IgE CL-5I | PTA-5680 | Dec. 3, 2003 |

This deposit was made under the provisions of the Budapest Treaty on the International Recognition of the Deposit of Microorganisms for the Purpose of Patent Procedure and the Regulations thereunder (Budapest Treaty). This assures maintenance of a viable culture for 30 years from the date of deposit. The organism will be made available by ATCC under the terms of the Budapest Treaty, which assures permanent and unrestricted availability of the progeny of the culture to the public upon issuance of the pertinent U.S. patent.

The assignee of the present application has agreed that if the culture on deposit should die or be lost or destroyed when cultivated under suitable conditions, it will be promptly replaced on notification with a viable specimen of the same culture. Availability of the deposited strain is not to be construed as a license to practice the invention in contravention of the rights granted under the authority of any government in accordance with its patent laws.

The foregoing written specification is considered to be sufficient to enable one skilled in the art to practice the invention. The present invention is not to be limited in scope by the cultures deposited, since the deposited embodiments are intended as illustration of one aspect of the invention and any culture that are functionally equivalent are within the scope of this invention. The deposit of material herein does not constitute an admission that the written description herein contained is inadequate to enable the practice of any aspect of the invention, including the best mode thereof, nor is it to be construed as limiting the scope of the claims to the specific illustration that it represents. Indeed, various modifications of the invention in addition to those shown and described herein will become apparent to those skilled in the art from the foregoing description and fall within the scope of the appended claims.

### Example 12

### Mapping of the High Affinity Binding Epitope of Human IgE

A. Peptide Synthesis and Anti-IgE Binding Assay

Studies have shown that IgE binds to its receptor through the C_{H}3 domain. Since the HA Anti-IgE antibodies of the present invention very efficiently block IgE from binding to its receptor, we mapped the epitope using peptides that encompassed the entire CH3 domain. First, we prepared two V5-tagged peptides, one comprised the entire constant region of human IgE and one comprised just the C_{H}2-C_{H}3 region of human IgE. These two peptides were expressed by in vitro transcription-translation and used in a Western blot assay to detect HA Anti-IgE MAb binding. Both CL-2C and CL-5I MAbs were able to bind to the intact human IgE as well as both the peptides.

To map the epitope more specifically, 73 overlapping peptides were synthesized which encompassed amino acid residues 141 to 368 of human IgE, which included the entire C_{H}3 domain. Each peptide consisted of 12 amino acid residues having a 3 amino acid overlap with the 3′ end of the previous peptide. SPOTs membranes were synthesized with fluorenylmethoxycarboyl (Fmoc) amino acids on cellulose membrane. The membranes were rinsed in methanol and then washed in TBS (pH 7.5) 3× for 10 min. After an overnight incubation in blocking solution (5% milk or 3% BSA in TBS), HRP-labeled anti-IgE MAbs diluted in blocking solution were incubated with the membrane for 3 hrs. After washing 3× for 15 min in TBS-TWEEN®, using SuperSignal HRP substrate (Pierce), IgE reactivity was measured by chemiluminescence exposure of BioMax MS film (Kodak) for the desired time.

The results from the experiment indicate that the HA Anti-IgE MAbs bind to two regions in the C_{H}3 portion of IgE, which are represented by the following two peptide sequences: NPRGVSAYLSRP (epitope “A”) and HPHLPRALMRST (Epitope “B”). (See FIG. 12) Binding to epitope A was several times weaker than to epitope B.

B. Alanine Scan Mutagenesis

An alanine scan along with substitutions of amino acids in the peptides with those that are found in IgG1 was carried out to determine which amino acids are critically involved in HA Anti-IgE MAb's binding to these peptides. Amino acids that were determined to be important for HA Anti-IgE MAb binding were replaced using an in vitro mutagenesis strategy in the ε chain of IgE. Another peptide covering the Cε2 and Cε3 regions as described earlier was also used in this study. (See FIGS. 13 and 14).

The EU numbering scheme for human IgE amino acid residues has been used. Polymerase chain reaction (PCR) was used to amplify the entire Fc region of IgE, and a truncated form of IgE Fc containing only the CH2-CH3 domain. The DNA products were cloned directly into pcDNA3 expression vector (Invitrogene, Carlsbad, Calif.) using TOPO cloning (Invitrogene, Carlsbad, Calif.).

Mutagenesis in IgE-Fc was performed using overlapping PCR (Ho et al., 1989). The DNA products were purified by agarose gel electrophoresis, digested with an appropriate restriction enzyme(s), and subcloned into the pcDNA3 expression vector. For each variant construct, PCR amplified regions were completely sequenced using dideoxynucleotide method from both strands of DNA. Recombinant human IgE Fc and its mutants were expressed using reticulocyte lysate based in-vitro transcription and translation coupled system (Promega, Madison, Wis.)

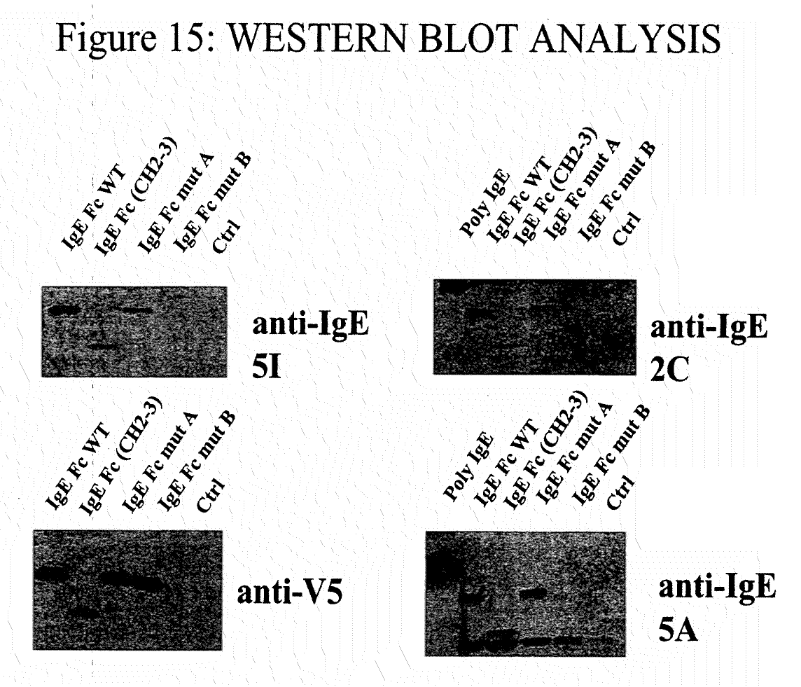

Lysates from this in-vitro transcription and translation coupled system (10 ul reaction mix) were subjected to SDS-PAGE (12%) and then transferred to nitrocellulalose membranes. The membranes were blocked with 5% dry milk in Tris-buffered saline (TBS) and subsequently stained with the primary antibody, anti-IgE MAbs. Specific reactive bands were detected using a goat anti-human IgG Fc conjugated to horseradish peroxidase (Jackson Labs, Bar Harbor, Me.) and the immunoreactive bands were visualized by the SuperSignal Western blotting detection kit (Pierce). Anti-V5 antibodies were used as a positive control that detected the V5 tag introduced at the C-terminus of these peptides. The Western blot with anti-V5 antibodies demonstrated that all the peptides were expressed at almost equal level. Interestingly, HA Anti-IgE MAbs were able to bind to the peptide that carried mutations in epitope ‘A’, but, they did not bind to the peptide that carried mutations in epitope ‘B’, indicating that this second site was more important for binding. (See FIG. 15).

### Example 13

### Active Immunization of Transgenic Mice Using an Immunogenic Peptide of Epitope B

Transgenic mice that constitutively express human IgE were used to demonstrate the active production of antibodies to a human immunogenic peptide of Epitope B. Two fusion peptides, each comprising an immunogenic peptide of the invention, a cysteine residue and KLH, were chemically synthesized. The sequence of peptide 1 was:

(KLH-Cys)-Leu Pro Arg Ala Leu Met Arg Ser Thr

and the sequence of peptide 2 was:

Leu Pro Arg Ala Leu Met Arg Ser Thr-(Cys-KLH)

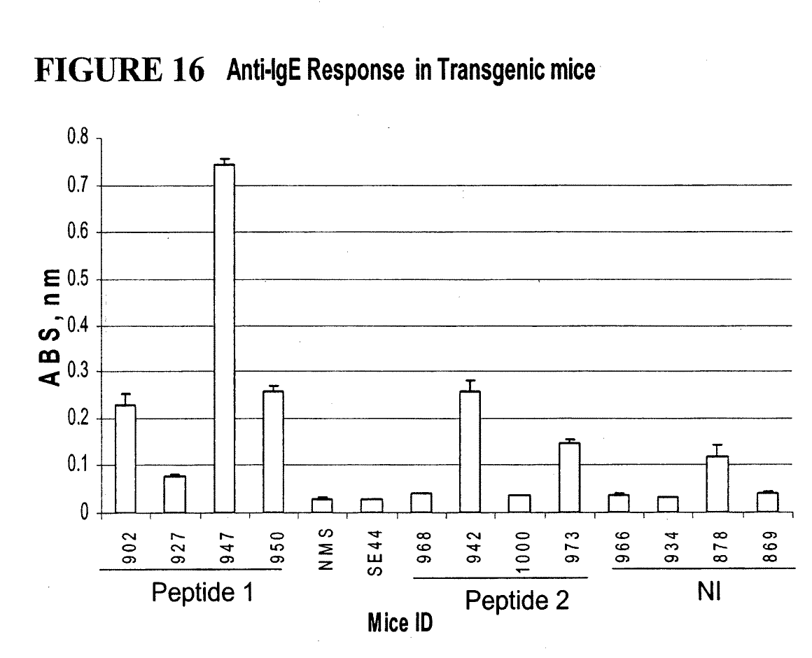

The transgenic mice were injected subcutaneously with 20 μg of the immunogenic peptide in complete Freund's adjuvant (Difco Laboratories, Detroit, Mich.) in 200 μL of PBS pH 7.4. At two-week intervals the mice were twice injected subcutaneously with 20 μg of the peptide immunogen in incomplete Freund's adjuvant. Then, two weeks later and three days prior to sacrifice; the mice were again injected intraperitoneally with 20 μg of the same immunogen in PBS. Serum was collected and tested for the presence of anti-IgE antibodies specific for Epitope B. As seen in FIG. 16, the peptide elicited anti-IgE antibodies in these transgenic mice.

Those skilled in the art will recognize, or be able to ascertain using no more than routine experimentation, many equivalents to the specific embodiments of the invention described herein. Such equivalents are intended to be encompassed by the following claims.

1-24. (canceled)

25. An isolated antibody that specifically binds to an epitope consisting of the amino acid sequence:
(a) Leu Pro Arg Ala Leu Xaa Arg Ser Xaa (SEQ ID NO 74),
(b) Leu Pro Arg Ala Leu Met Arg Ser Thr (SEQ ID NO 75),
(c) His Pro H is Leu Pro Arg Ala Leu Met Arg Ser Thr (SEQ ID NO 76), or
(d) Leu Pro Arg Ala Leu Met Arg Ser Thr Thr Lys Thr (SEQ ID NO 77).

26. The antibody of claim 25, further comprising a label.

27. The antibody of claim 25, wherein the antibody is:
a) a chimeric antibody,
b) a single chain antibody,
c) a Fab fragment,
d) a F(ab′) fragment,
e) a human antibody, or
f) a humanized antibody.

28. A composition comprising the antibody of claim 25 and an acceptable carrier.

29. The antibody of claim 25, wherein the antibody is a polyclonal antibody.

30. A composition comprising the polyclonal antibody of claim 29 and a suitable carrier.

31. The antibody of claim 25, wherein the antibody is a monoclonal antibody.

32. A composition comprising the monoclonal antibody of claim 31 and a suitable carrier.

33. The antibody of claim 25, wherein the antibody is produced by screening a Fab expression library.

34. The antibody of claim 25, wherein the antibody is produced by screening a combinatorial immunoglobulin library.

35. A kit comprising the antibody of claim 25.
